SEQUENCE LISTING (307 sequences)

SEQ ID NO: 1 - protein, 8 aa, synthetic construct
GFTFSDYY

SEQ ID NO: 2 - protein, 9 aa, synthetic construct
YISSSGSTI

SEQ ID NO: 3 - protein, 15 aa, synthetic construct
ARVGGDFWSGFLMDV

SEQ ID NO: 4 - protein, 25 aa, synthetic construct
QVQLVESGGGLVKPGGSLRLSCAAS

SEQ ID NO: 5 - protein, 16 aa, synthetic construct
MSWIRQAPGKGLEWVS

SEQ ID NO: 6 - protein, 38 aa, synthetic construct
YYADSVKGRFTISRDNAKNSLYLQMNSLRAEDTAVYYC

SEQ ID NO: 7 - protein, 11 aa, synthetic construct
WGQGTTVTVSS

SEQ ID NO: 8 - protein, 122 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVGGDFWSGFLMDVWGQGTTVTVSS

SEQ ID NO: 9 - protein, 11 aa, synthetic construct
RASQSISSYLN

SEQ ID NO: 10 - protein, 7 aa, synthetic construct
AASSLQS

SEQ ID NO: 11 - protein, 9 aa, synthetic construct
MQALQTPLT

SEQ ID NO: 12 - protein, 23 aa, synthetic construct
DIQMTQSPSSLSASVGDRVTITC

SEQ ID NO: 13 - protein, 15 aa, synthetic construct
WYQQKPGKAPKLLIY

SEQ ID NO: 14 - protein, 32 aa, synthetic construct
GVPSRFSGSGSGTDFTLTISSLQPEDVAVYYC

SEQ ID NO: 15 - protein, 11 aa, synthetic construct
FGQGTKLEIKR

SEQ ID NO: 16 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDVAVYYCMQALQTPLTFGQGTKLEIKR

SEQ ID NO: 17 - protein, 8 aa, synthetic construct
GFTFSNYA

SEQ ID NO: 18 - protein, 9 aa, synthetic construct
VISYDGSNK

SEQ ID NO: 19 - protein, 12 aa, synthetic construct
AKDLPWEWELDY

SEQ ID NO: 20 - protein, 25 aa, synthetic construct
EVQLVESGGGVVQPGRSLRLSCAAS

SEQ ID NO: 21 - protein, 16 aa, synthetic construct
MHWVRQAPGKGLEWVA

SEQ ID NO: 22 - protein, 38 aa, synthetic construct
YYADSVKGRFTISRDDSTNTLYLQMNNLRAEDTAVYYC

SEQ ID NO: 23 - protein, 11 aa, synthetic construct
WGQGTLVTVSS

SEQ ID NO: 24 - protein, 119 aa, synthetic construct
EVQLVESGGGVVQPGRSLRLSCAASGFTFSNYAMHWVRQAPGKGLEWVAVISYDGSNKYYADSVKGRFTISRDDSTNTLYLQMNNLRAEDTAVYYCAKDLPWEWELDYWGQGTLVTVSS

SEQ ID NO: 25 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 26 - protein, 8 aa, synthetic construct
EPKSCDKT

SEQ ID NO: 27 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - DNA, 366 bp, synthetic construct
  source 1..366: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggctgtgtattactgtgcgagggtaggtggcgatttttggagtggtttccttatggacgtctggggccaagggaccacggtcaccgtctcctca

SEQ ID NO: 29 - DNA, 324 bp, synthetic construct
  source 1..324: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgtggcagtttattactgcatgcaagctctacaaactcctctgacgttcggccaagggaccaaactggagatcaaacgt

SEQ ID NO: 30 - DNA, 357 bp, synthetic construct
  source 1..357: mol_type = other DNA
gaggtgcagctggtggagtccgggggaggcgtggtccagcctgggaggtccctgagactctcctgtgcagcctctggattcaccttcagtaactatgctatgcactgggtccgccaggctccaggcaaggggctggagtgggtggcagttatatcatatgatggaagcaataaatactacgcagactccgtgaagggccgattcaccatctccagagacgattccacgaacacgctgtatctgcaaatgaacaacctgagagccgaggacacagccgtatattactgtgcgaaagatcttccatgggagtgggagctagactactggggccagggcaccctggtcaccgtctcctca

SEQ ID NO: 31 - protein, 15 aa, synthetic construct
EPKSCDKTHTCPPCP

SEQ ID NO: 32 - DNA, 45 bp, synthetic construct
  source 1..45: mol_type = other DNA
gagcccaaatcttgtgacaaaactcacacatgcccaccgtgccca

SEQ ID NO: 33 - DNA, 651 bp, synthetic construct
  source 1..651: mol_type = other DNA
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacacctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 34 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 1356 bp, synthetic construct
  source 1..1356: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggctgtgtattactgtgcgagggtaggtggcgatttttggagtggtttccttatggacgtctggggccaagggaccacggtcaccgtctcctcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctccagcagcttgggcacccagacctacatctgcaacgtgaatcacaagcccagcaacaccaaggtcgacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacacctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 36 - protein, 452 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVGGDFWSGFLMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 642 bp, synthetic construct
  source 1..642: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgtggcagtttattactgcatgcaagctctacaaactcctctgacgttcggccaagggaccaaactggagatcaaacgtacggtggctgctccgtcggtgtttatctttccaccgagtgacgaacaactgaagtcggggactgccagcgttgtttgccttctgaacaatttttacccccgcgaggcgaaggttcagtggaaagtcgataatgcgttacagagcgggaatagccaggaaagtgttactgaacaggatagtaaagacagcacttacagcctcagcagcaccctgacgctgagcaaagcagactacgaaaaacataaagtctacgcctgcgaagtcacccatcagggcctgagctcgcccgtcacaaagagcttcaacaggggagagtgt

SEQ ID NO: 38 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDVAVYYCMQALQTPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 1044 bp, synthetic construct
  source 1..1044: mol_type = other DNA
gaggtgcagctggtggagtccgggggaggcgtggtccagcctgggaggtccctgagactctcctgtgcagcctctggattcaccttcagtaactatgctatgcactgggtccgccaggctccaggcaaggggctggagtgggtggcagttatatcatatgatggaagcaataaatactacgcagactccgtgaagggccgattcaccatctccagagacgattccacgaacacgctgtatctgcaaatgaacaacctgagagccgaggacacagccgtatattactgtgcgaaagatcttccatgggagtgggagctagactactggggccagggcaccctggtcaccgtctcctcatccggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacacctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 40 - protein, 348 aa, synthetic construct
EVQLVESGGGVVQPGRSLRLSCAASGFTFSNYAMHWVRQAPGKGLEWVAVISYDGSNKYYADSVKGRFTISRDDSTNTLYLQMNNLRAEDTAVYYCAKDLPWEWELDYWGQGTLVTVSSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41-42 - skipped (2 entries)

SEQ ID NO: 43 - protein, 255 aa, synthetic construct
DIQMTQSPSSLSASVGDRVTITCRASQSISSYLNWYQQKPGKAPKLLIYAASSLQSGVPSRFSGSGSGTDFTLTISSLQPEDVAVYYCMQALQTPLTFGQGTKLEIKRGGGGSGGGGSGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVGGDFWSGFLMDVWGQGTTVTVSS

SEQ ID NO: 44 - protein, 245 aa, synthetic construct
DIQMTQSPSSLSASVGDRVTITCRASQSISSYLNWYQQKPGKAPKLLIYAASSLQSGVPSRFSGSGSGTDFTLTISSLQPEDVAVYYCMQALQTPLTFGQGTKLEIKRGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVGGDFWSGFLMDVWGQGTTVTVSS

SEQ ID NO: 45 - protein, 255 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVGGDFWSGFLMDVWGQGTTVTVSSGGGGSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDVAVYYCMQALQTPLTFGQGTKLEIKR

SEQ ID NO: 46 - protein, 245 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVGGDFWSGFLMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDVAVYYCMQALQTPLTFGQGTKLEIKR

SEQ ID NO: 47 - protein, 15 aa, synthetic construct
GGGGSGGSGSGGGGS

SEQ ID NO: 48 - protein, 15 aa, synthetic construct
GGGGSGGSGSSGGGS

SEQ ID NO: 49 - protein, 15 aa, synthetic construct
GGGGSGGGGSGGGGS

SEQ ID NO: 50 - protein, 15 aa, synthetic construct
GGGGSGSGASSGGGS

SEQ ID NO: 51 - protein, 15 aa, synthetic construct
GGGGSGSGASGGGGS

SEQ ID NO: 52 - protein, 15 aa, synthetic construct
SGGGSGGGGSGGGGS

SEQ ID NO: 53 - protein, 18 aa, synthetic construct
GSTSGSGKPGSGEGSTKG

SEQ ID NO: 54 - protein, 19 aa, synthetic construct
PNGASQSSSASHTGSAPGS

SEQ ID NO: 55 - protein, 19 aa, synthetic construct
PNGASNSGSAPDTSSAPGS

SEQ ID NO: 56 - protein, 19 aa, synthetic construct
PNGASHSGSAPNTSSAPGS

SEQ ID NO: 57 - protein, 19 aa, synthetic construct
PNGASESGSASKTSSASGS

SEQ ID NO: 58 - protein, 19 aa, synthetic construct
PNGASNSGSAPKTGSASGS

SEQ ID NO: 59 - protein, 19 aa, synthetic construct
PNGASKSGSASQTSSAPGS

SEQ ID NO: 60 - protein, 19 aa, synthetic construct
PNGASHSSSASQTGSAPGS

SEQ ID NO: 61 - protein, 12 aa, synthetic construct
PNGASKRSAPGS

SEQ ID NO: 62 - protein, 19 aa, synthetic construct
PNGASHSGSAPHTSSASGS

SEQ ID NO: 63 - protein, 15 aa, synthetic construct
RGRGRGRGRSRGGGS

SEQ ID NO: 64 - protein, 15 aa, synthetic construct
SHGGSHGGGSGGGGS

SEQ ID NO: 65 - protein, 5 aa, synthetic construct
GQAGR

SEQ ID NO: 66 - protein, 8 aa, synthetic construct
GGGSGGGG

SEQ ID NO: 67 - protein, 5 aa, synthetic construct
SGGGG

SEQ ID NO: 68 - protein, 5 aa, synthetic construct
GGGGS

SEQ ID NO: 69 - protein, 30 aa, synthetic construct
GGGGSGGGGSGGGGSGGGGSGGGGSGGGGS

SEQ ID NO: 70 - protein, 16 aa, synthetic construct
GGGGGSGGGGSGGGGS

SEQ ID NO: 71 - protein, 26 aa, synthetic construct
GGGGGSGGGGSGGGGSGGGGSGGGGS

SEQ ID NO: 72 - protein, 25 aa, synthetic construct
GGGGSGGGGSGGGGSGGGGSGGGGS

SEQ ID NO: 73 - skipped

SEQ ID NO: 74 - protein, 267 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 75 - protein, 218 aa, Homo sapiens [sequence omitted]

SEQ ID NO: 76 - protein, 8 aa, synthetic construct
GFTFSSYY

SEQ ID NO: 77 - protein, 8 aa, synthetic construct
GFTFSNHY

SEQ ID NO: 78 - protein, 8 aa, synthetic construct
GYTFTDYY

SEQ ID NO: 79 - protein, 8 aa, synthetic construct
GFTFNDHY

SEQ ID NO: 80 - protein, 8 aa, synthetic construct
GFTFSDYF

SEQ ID NO: 81 - protein, 8 aa, synthetic construct
GFTFSEYY

SEQ ID NO: 82 - protein, 8 aa, synthetic construct
GFTFTDYY

SEQ ID NO: 83 - protein, 8 aa, synthetic construct
GFTFGDYY

SEQ ID NO: 84 - protein, 8 aa, synthetic construct
GFTFADYY

SEQ ID NO: 85 - protein, 8 aa, synthetic construct
GFSFSDYY

SEQ ID NO: 86 - protein, 9 aa, synthetic construct
YISSSGNTI

SEQ ID NO: 87 - protein, 9 aa, synthetic construct
YISNSGSTI

SEQ ID NO: 88 - protein, 9 aa, synthetic construct
YVSSSGSTI

SEQ ID NO: 89 - protein, 9 aa, synthetic construct
YISSSGTTI

SEQ ID NO: 90 - protein, 9 aa, synthetic construct
YIASSGSTI

SEQ ID NO: 91 - protein, 9 aa, synthetic construct
YISSTGSTI

SEQ ID NO: 92 - protein, 9 aa, synthetic construct
YISSSGSAI

SEQ ID NO: 93 - protein, 9 aa, synthetic construct
YINSSGSTI

SEQ ID NO: 94 - protein, 9 aa, synthetic construct
YISSSSSTI

SEQ ID NO: 95 - protein, 9 aa, synthetic construct
YIGSSGSTI

SEQ ID NO: 96 - protein, 15 aa, synthetic construct
ARVGGDFWSGYLMDV

SEQ ID NO: 97 - protein, 15 aa, synthetic construct
ARVGGDFWSGYFMDV

SEQ ID NO: 98 - protein, 15 aa, synthetic construct
ARVGSDFWSGFRMDV

SEQ ID NO: 99 - protein, 15 aa, synthetic construct
AREGGDFWSGFLMDV

SEQ ID NO: 100 - protein, 15 aa, synthetic construct
ARGGNDFWSGYLMDV

SEQ ID NO: 101 - protein, 15 aa, synthetic construct
ARVGNDFWSGYWMDV

SEQ ID NO: 102 - protein, 15 aa, synthetic construct
ARTEGDFWSGYLMDV

SEQ ID NO: 103 - protein, 15 aa, synthetic construct
ARGGGDFWSGYMMDV

SEQ ID NO: 104 - protein, 15 aa, synthetic construct
ARMGDDFWSGYLMDV

SEQ ID NO: 105 - protein, 15 aa, synthetic construct
ARGGGDFWRGYYMDV

SEQ ID NO: 106 - protein, 11 aa, synthetic construct
RASQSVSSYLN

SEQ ID NO: 107 - protein, 11 aa, synthetic construct
RASQSISTYLN

SEQ ID NO: 108 - protein, 11 aa, synthetic construct
RASQSISNYLN

SEQ ID NO: 109 - protein, 11 aa, synthetic construct
RASQSITSYLN

SEQ ID NO: 110 - protein, 11 aa, synthetic construct
RASQSISAYLN

SEQ ID NO: 111 - protein, 11 aa, synthetic construct
RASQSINSYLN

SEQ ID NO: 112 - protein, 11 aa, synthetic construct
RASQTISSYLN

SEQ ID NO: 113 - protein, 11 aa, synthetic construct
RSSQSISSYLN

SEQ ID NO: 114 - protein, 11 aa, synthetic construct
RASQSISSYLD

SEQ ID NO: 115 - protein, 11 aa, synthetic construct
RASQSIASYLN

SEQ ID NO: 116 - protein, 7 aa, synthetic construct
AAASLQS

SEQ ID NO: 117 - protein, 7 aa, synthetic construct
AASALQS

SEQ ID NO: 118 - protein, 7 aa, synthetic construct
AASTLQS

SEQ ID NO: 119 - protein, 7 aa, synthetic construct
AASNLQS

SEQ ID NO: 120 - protein, 7 aa, synthetic construct
ASSSLQS

SEQ ID NO: 121 - protein, 7 aa, synthetic construct
AANSLQS

SEQ ID NO: 122 - protein, 7 aa, synthetic construct
AASSMQS

SEQ ID NO: 123 - protein, 7 aa, synthetic construct
AANSMQS

SEQ ID NO: 124 - protein, 7 aa, synthetic construct
AAASMQS

SEQ ID NO: 125 - protein, 7 aa, synthetic construct
ASSTLQS

SEQ ID NO: 126 - protein, 9 aa, synthetic construct
MQALQSPLT

SEQ ID NO: 127 - protein, 9 aa, synthetic construct
MQALHTPLT

SEQ ID NO: 128 - protein, 9 aa, synthetic construct
MQTLQTPLT

SEQ ID NO: 129 - protein, 9 aa, synthetic construct
MQALETPLT

SEQ ID NO: 130 - protein, 9 aa, synthetic construct
MQALQAPLT

SEQ ID NO: 131 - protein, 9 aa, synthetic construct
MQALQNPLT

SEQ ID NO: 132 - protein, 9 aa, synthetic construct
MQAIQTPLT

SEQ ID NO: 133 - protein, 9 aa, synthetic construct
MQVLQTPLT

SEQ ID NO: 134 - protein, 9 aa, synthetic construct
MQALRTPLT

SEQ ID NO: 135 - protein, 9 aa, synthetic construct
MQALQTQLT

SEQ ID NO: 136 - protein, 8 aa, synthetic construct
GFSFSNYA

SEQ ID NO: 137 - protein, 8 aa, synthetic construct
GFTFANYA

SEQ ID NO: 138 - protein, 8 aa, synthetic construct
GFTFNNYA

SEQ ID NO: 139 - protein, 8 aa, synthetic construct
GFTFSNSA

SEQ ID NO: 140 - protein, 8 aa, synthetic construct
GFTFTNYA

SEQ ID NO: 141 - protein, 8 aa, synthetic construct
GFTFSDYA

SEQ ID NO: 142 - protein, 8 aa, synthetic construct
GFTYSNYA

SEQ ID NO: 143 - protein, 8 aa, synthetic construct
GYTFSNYA

SEQ ID NO: 144 - protein, 8 aa, synthetic construct
GFTFGNYA

SEQ ID NO: 145 - protein, 8 aa, synthetic construct
GFTFSHYA

SEQ ID NO: 146 - protein, 9 aa, synthetic construct
VISFDGSNK

SEQ ID NO: 147 - protein, 9 aa, synthetic construct
VISYNGSNK

SEQ ID NO: 148 - protein, 9 aa, synthetic construct
VVSYDGSNK

SEQ ID NO: 149 - protein, 9 aa, synthetic construct
VISYDGTNK

SEQ ID NO: 150 - protein, 9 aa, synthetic construct
VIAYDGSNK

SEQ ID NO: 151 - protein, 9 aa, synthetic construct
VISYDGSDK

SEQ ID NO: 152 - protein, 9 aa, synthetic construct
VISYEGSNK

SEQ ID NO: 153 - protein, 9 aa, synthetic construct
VISYDGNNK

SEQ ID NO: 154 - protein, 9 aa, synthetic construct
VISYDGANK

SEQ ID NO: 155 - protein, 9 aa, synthetic construct
VITYDGSNK

SEQ ID NO: 156 - protein, 12 aa, synthetic construct
AKILPWEKELDY

SEQ ID NO: 157 - protein, 12 aa, synthetic construct
AEDLPWSWDLDY

SEQ ID NO: 158 - protein, 12 aa, synthetic construct
AKDLSWNWELDF

SEQ ID NO: 159 - protein, 12 aa, synthetic construct
AKALAWEWELEY

SEQ ID NO: 160 - protein, 12 aa, synthetic construct
AKNLPWEWELDY

SEQ ID NO: 161 - protein, 12 aa, synthetic construct
AKDPPWGWELDY

SEQ ID NO: 162 - protein, 12 aa, synthetic construct
AKKLPWEWELDY

SEQ ID NO: 163 - protein, 12 aa, synthetic construct
AMDLSWDWALNY

SEQ ID NO: 164 - protein, 12 aa, synthetic construct
AKELPWQWEEDY

SEQ ID NO: 165 - protein, 12 aa, synthetic construct
AKDLSWQWELEF

SEQ ID NO: 166 - protein, 240 aa, Homo sapiens
SEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 167 - protein, 7 aa, synthetic construct
RPHAWPF

SEQ ID NO: 168 - protein, 13 aa, synthetic construct
VRVVLGAHNLSRR

SEQ ID NO: 169 - protein, 6 aa, synthetic construct
FENGYD

SEQ ID NO: 170 - protein, 12 aa, synthetic construct
VQVAQLPAQGRR

SEQ ID NO: 171 - protein, 13 aa, synthetic construct
VRVVLGAHNLSRR

SEQ ID NO: 172 - protein, 12 aa, synthetic construct
VQVAQLPAQGRR

SEQ ID NO: 173 - protein, 12 aa, synthetic construct
RRSNVCTLVRGR

SEQ ID NO: 174 - protein, 7 aa, synthetic construct
RPHAWPF

SEQ ID NO: 175 - protein, 6 aa, synthetic construct
FENGYD

SEQ ID NO: 176 - protein, 7 aa, synthetic construct
RPHAWPF

SEQ ID NO: 177 - protein, 7 aa, synthetic construct
IFENGYD

SEQ ID NO: 178 - protein, 7 aa, synthetic construct
GSGSGSG

SEQ ID NO: 179 - protein, 25 aa, synthetic construct
GGGGSGGGGSGGGGSGGGGSGGGGS

SEQ ID NO: 180 - protein, 15 aa, synthetic construct
GGGGSGGGGSGGGGS

SEQ ID NO: 181 - protein, 7 aa, synthetic construct
GSGSGSG

SEQ ID NO: 182 - protein, 7 aa, synthetic construct
PFMVSLQ

SEQ ID NO: 183 - protein, 7 aa, synthetic construct
CGATLIA

SEQ ID NO: 184 - protein, 7 aa, synthetic construct
AAHCVAN

SEQ ID NO: 185 - protein, 7 aa, synthetic construct
RVVLGAH

SEQ ID NO: 186 - protein, 7 aa, synthetic construct
PTRQVFA

SEQ ID NO: 187 - protein, 7 aa, synthetic construct
NGYDPVN

SEQ ID NO: 188 - protein, 7 aa, synthetic construct
ILQLNGS

SEQ ID NO: 189 - protein, 7 aa, synthetic construct
VQVAQLP

SEQ ID NO: 190 - protein, 7 aa, synthetic construct
GNGVQCL

SEQ ID NO: 191 - protein, 7 aa, synthetic construct
LGRNRGI

SEQ ID NO: 192 - protein, 7 aa, synthetic construct
LNVTVVT

SEQ ID NO: 193 - protein, 7 aa, synthetic construct
NVCTLVR

SEQ ID NO: 194 - protein, 7 aa, synthetic construct
CFGDSGS

SEQ ID NO: 195 - protein, 7 aa, synthetic construct
LIHGIAS

SEQ ID NO: 196 - protein, 7 aa, synthetic construct
ASGLYPD

SEQ ID NO: 197 - protein, 7 aa, synthetic construct
QFVNWID

SEQ ID NO: 198 - protein, 10 aa, synthetic construct
RARPHAWPFM

SEQ ID NO: 199 - protein, 10 aa, synthetic construct
QLRGGHFCGA

SEQ ID NO: 200 - protein, 10 aa, synthetic construct
APNFVMSAAH

SEQ ID NO: 201 - protein, 10 aa, synthetic construct
NVNVRAVRVV

SEQ ID NO: 202 - protein, 10 aa, synthetic construct
HNLSRREPTR

SEQ ID NO: 203 - protein, 10 aa, synthetic construct
AVQRIFENGY

SEQ ID NO: 204 - protein, 10 aa, synthetic construct
NLLNDIVILQ

SEQ ID NO: 205 - protein, 10 aa, synthetic construct
SATINANVQV

SEQ ID NO: 206 - protein, 10 aa, synthetic construct
PAQGRRLGNG

SEQ ID NO: 207 - protein, 10 aa, synthetic construct
LAMGWGLLGR

SEQ ID NO: 208 - protein, 10 aa, synthetic construct
IASVLQELNV

SEQ ID NO: 209 - protein, 10 aa, synthetic construct
TSLCRRSNVC

SEQ ID NO: 210 - protein, 10 aa, synthetic construct
RGRQAGVCFG

SEQ ID NO: 211 - protein, 10 aa, synthetic construct
SPLVCNGLIH

SEQ ID NO: 212 - protein, 10 aa, synthetic construct
SFVRGGCASG

SEQ ID NO: 213 - protein, 10 aa, synthetic construct
DAFAPVAQFV

SEQ ID NO: 214 - protein, 13 aa, synthetic construct
VGGRRARPHAWPF

SEQ ID NO: 215 - protein, 13 aa, synthetic construct
MVSLQLRGGHFCG

SEQ ID NO: 216 - protein, 13 aa, synthetic construct
ATLIAPNFVMSAA

SEQ ID NO: 217 - protein, 13 aa, synthetic construct
HCVANVNVRAVRV

SEQ ID NO: 218 - protein, 13 aa, synthetic construct
VLGAHNLSRREPT

SEQ ID NO: 219 - protein, 13 aa, synthetic construct
RQVFAVQRIFENG

SEQ ID NO: 220 - protein, 13 aa, synthetic construct
YDPVNLLNDIVIL

SEQ ID NO: 221 - protein, 13 aa, synthetic construct
QLNGSATINANVQ

SEQ ID NO: 222 - protein, 13 aa, synthetic construct
VAQLPAQGRRLGN

SEQ ID NO: 223 - protein, 13 aa, synthetic construct
GVQCLAMGWGLLG

SEQ ID NO: 224 - protein, 13 aa, synthetic construct
RNRGIASVLQELN

SEQ ID NO: 225 - protein, 13 aa, synthetic construct
VTVVTSLCRRSNV

SEQ ID NO: 226 - protein, 13 aa, synthetic construct
CTLVRGRQAGVCF

SEQ ID NO: 227 - protein, 13 aa, synthetic construct
GDSGSPLVCNGLI

SEQ ID NO: 228 - protein, 13 aa, synthetic construct
HGIASFVRGGCAS

SEQ ID NO: 229 - protein, 13 aa, synthetic construct
GLYPDAFAPVAQF

SEQ ID NO: 230 - protein, 6 aa, synthetic construct
PHAWPF

SEQ ID NO: 231 - protein, 7 aa, synthetic construct
AHNLSRR

SEQ ID NO: 232 - protein, 4 aa, synthetic construct
ENGY

SEQ ID NO: 233 - protein, 6 aa, synthetic construct
PAQGRR

SEQ ID NO: 234 - protein, 7 aa, synthetic construct
RPHAWPF

SEQ ID NO: 235 - protein, 10 aa, synthetic construct
VLGAHNLSRR

SEQ ID NO: 236 - protein, 5 aa, synthetic construct
FENGY

SEQ ID NO: 237 - protein, 9 aa, synthetic construct
AQLPAQGRR

SEQ ID NO: 238 - protein, 5 aa, synthetic construct
HAWPF

SEQ ID NO: 239 - protein, 13 aa, synthetic construct
VRVVLGAHNLSRR

SEQ ID NO: 240 - protein, 6 aa, synthetic construct
FENGYD

SEQ ID NO: 241 - protein, 12 aa, synthetic construct
VQVAQLPAQGRR

SEQ ID NO: 242 - protein, 12 aa, synthetic construct
RRSNVCTLVRGR

SEQ ID NO: 243 - protein, 7 aa, synthetic construct
GSGSGSG

SEQ ID NO: 244 - protein, 7 aa, synthetic construct
PFMVSLQ

SEQ ID NO: 245 - protein, 7 aa, synthetic construct
CGATLIA

SEQ ID NO: 246 - protein, 7 aa, synthetic construct
AAHCVAN

SEQ ID NO: 247 - protein, 7 aa, synthetic construct
RVVLGAH

SEQ ID NO: 248 - protein, 7 aa, synthetic construct
PTRQVFA

SEQ ID NO: 249 - protein, 7 aa, synthetic construct
NGYDPVN

SEQ ID NO: 250 - protein, 7 aa, synthetic construct
ILQLNGS

SEQ ID NO: 251 - protein, 7 aa, synthetic construct
VQVAQLP

SEQ ID NO: 252 - protein, 7 aa, synthetic construct
GNGVQCL

SEQ ID NO: 253 - protein, 7 aa, synthetic construct
LGRNRGI

SEQ ID NO: 254 - protein, 7 aa, synthetic construct
LNVTVVT

SEQ ID NO: 255 - protein, 7 aa, synthetic construct
NVCTLVR

SEQ ID NO: 256 - protein, 7 aa, synthetic construct
CFGDSGS

SEQ ID NO: 257 - protein, 7 aa, synthetic construct
LIHGIAS

SEQ ID NO: 258 - protein, 7 aa, synthetic construct
ASGLYPD

SEQ ID NO: 259 - protein, 7 aa, synthetic construct
QFVNWID

SEQ ID NO: 260 - protein, 10 aa, synthetic construct
RARPHAWPFM

SEQ ID NO: 261 - protein, 10 aa, synthetic construct
QLRGGHFCGA

SEQ ID NO: 262 - protein, 10 aa, synthetic construct
APNFVMSAAH

SEQ ID NO: 263 - protein, 10 aa, synthetic construct
NVNVRAVRVV

SEQ ID NO: 264 - protein, 10 aa, synthetic construct
HNLSRREPTR

SEQ ID NO: 265 - protein, 10 aa, synthetic construct
AVQRIFENGY

SEQ ID NO: 266 - protein, 10 aa, synthetic construct
NLLNDIVILQ

SEQ ID NO: 267 - protein, 10 aa, synthetic construct
SATINANVQV

SEQ ID NO: 268 - protein, 10 aa, synthetic construct
PAQGRRLGNG

SEQ ID NO: 269 - protein, 10 aa, synthetic construct
LAMGWGLLGR

SEQ ID NO: 270 - protein, 10 aa, synthetic construct
IASVLQELNV

SEQ ID NO: 271 - protein, 10 aa, synthetic construct
TSLCRRSNVC

SEQ ID NO: 272 - protein, 10 aa, synthetic construct
RGRQAGVCFG

SEQ ID NO: 273 - protein, 10 aa, synthetic construct
SPLVCNGLIH

SEQ ID NO: 274 - protein, 10 aa, synthetic construct
SFVRGGCASG

SEQ ID NO: 275 - protein, 10 aa, synthetic construct
DAFAPVAQFV

SEQ ID NO: 276 - protein, 13 aa, synthetic construct
IVGGRRARPHAWP

SEQ ID NO: 277 - protein, 13 aa, synthetic construct
FMVSLQLRGGHFC

SEQ ID NO: 278 - protein, 13 aa, synthetic construct
GATLIAPNFVMSA

SEQ ID NO: 279 - protein, 13 aa, synthetic construct
AHCVANVNVRAVR

SEQ ID NO: 280 - protein, 13 aa, synthetic construct
VVLGAHNLSRREP

SEQ ID NO: 281 - protein, 13 aa, synthetic construct
TRQVFAVQRIFEN

SEQ ID NO: 282 - protein, 13 aa, synthetic construct
GYDPVNLLNDIVI

SEQ ID NO: 283 - protein, 13 aa, synthetic construct
LQLNGSATINANV

SEQ ID NO: 284 - protein, 13 aa, synthetic construct
QVAQLPAQGRRLG

SEQ ID NO: 285 - protein, 13 aa, synthetic construct
NGVQCLAMGWGLL

SEQ ID NO: 286 - protein, 13 aa, synthetic construct
GRNRGIASVLQEL

SEQ ID NO: 287 - protein, 13 aa, synthetic construct
NVTVVTSLCRRSN

SEQ ID NO: 288 - protein, 13 aa, synthetic construct
VCTLVRGRQAGVC

SEQ ID NO: 289 - protein, 13 aa, synthetic construct
FGDSGSPLVCNGL

SEQ ID NO: 290 - protein, 13 aa, synthetic construct
IHGIASFVRGGCA

SEQ ID NO: 291 - protein, 13 aa, synthetic construct
SGLYPDAFAPVAQ

SEQ ID NO: 292 - protein, 5 aa, synthetic construct
ENGYD

SEQ ID NO: 293 - protein, 5 aa, synthetic construct
VNLLN

SEQ ID NO: 294 - protein, 6 aa, synthetic construct
GVCFGD

SEQ ID NO: 295 - protein, 7 aa, synthetic construct
FVNVVID

SEQ ID NO: 296 - protein, 6 aa, synthetic construct
FENGYD

SEQ ID NO: 297 - protein, 6 aa, synthetic construct
VNLLND

SEQ ID NO: 298 - protein, 6 aa, synthetic construct
GVCFGD

SEQ ID NO: 299 - protein, 6 aa, synthetic construct
SGLYPD

SEQ ID NO: 300 - protein, 6 aa, synthetic construct
FVNWID

SEQ ID NO: 301 - protein, 6 aa, synthetic construct
IQRSED

SEQ ID NO: 302 - protein, 7 aa, synthetic construct
IFENGYD

SEQ ID NO: 303 - protein, 9 aa, synthetic construct
YDPVNLLND

SEQ ID NO: 304 - protein, 9 aa, synthetic construct
RQAGVCFGD

SEQ ID NO: 305 - protein, 9 aa, synthetic construct
GCASGLYPD

SEQ ID NO: 306 - protein, 10 aa, synthetic construct
PVAQFVNWID

SEQ ID NO: 307 - protein, 9 aa, synthetic construct
DSIIQRSED

(19) United States
(12) Patent Application Publication
(10) Pub. No.: US 2024/0392037 A1
(43) Pub. Date: Nov. 28, 2024

(54) MOLECULES THAT BIND TO NEUTROPHIL ELASTASE POLYPEPTIDES

(71) Applicant: University of Pittsburgh - of the Commonwealth System of Higher Education, Pittsburgh, PA (US)

(72) Inventors: Dimiter Stanchev Dimitrov, Pittsburgh, PA (US); Xiaojie Chu, Pittsburgh, PA (US); Steven David Shapiro, Pittsburgh, PA (US); John W. Mellors, Pittsburgh, PA (US); Zehua Sun, Pittsburgh, PA (US); Dusan Baek, Pittsburgh, PA (US)

(21) Appl. No.: 18/694,046

(22) PCT Filed: Jan. 10, 2023
(86) PCT No.: PCT/US23/10453

§ 371 Date: Mar. 21, 2024

Related U.S. Application Data
(60) Provisional application No. 63/297,886, filed on Jan. 10, 2022.

(51) Int. Cl. C07K 16/40; A61K 39/00; A61P 35/00

(52) U.S. Cl. CPC C07K 16/40; A61P 35/00; A61K 2039/505

(57) ABSTRACT

This document provides methods and materials involved in binding a binder (e.g., an antibody, antigen binding fragment, and/or antibody domain) to an NE polypeptide. For example, binders (e.g., antibodies, antigen binding fragments, and/or antibody domains) that bind to an NE polypeptide and methods and materials for using one or more such binding molecules to treat a mammal (e.g., a human) having cancer and/or an inflammatory condition are provided.

### CROSS-REFERENCE TO RELATED APPLICATIONS

This application claims the benefit of U.S. Provisional Application Ser. No. 63/297,886, filed Jan. 10, 2022. The disclosure of the prior application is considered part of (and is incorporated by reference in) the disclosure of this application.

### BACKGROUND

### 1. Technical Field

This document relates to methods and materials involved in binding a molecule (e.g., an antibody, a fragment of an antibody, or an antibody domain) to a neutrophil elastase (NE) polypeptide (e.g., a human NE polypeptide). For example, this document provides binders (e.g., antibodies, antigen binding fragments, and antibody domains) that bind to an NE polypeptide and methods and materials for using such binders to treat cancer and/or an inflammatory condition. This document also provides cells (e.g., host cells) designed to express one or more binders (e.g., antibodies, antigen binding fragments, or antibody domains) having the ability to bind to an NE polypeptide and methods and materials for using such cells to treat cancer and/or an inflammatory condition.

### 2. Background Information

Neutrophil elastase (NE) is a serine protease released by neutrophils, and it is generally considered a main contributor of neutrophil protease activity. Neutrophils are the most abundant white blood cells and play a major role in host defense against bacterial infection. They can rapidly release cytokines, chemokines, reactive oxygen species, and proteases to help defend against bacterial infections and regulate inflammation (Witter et al., J. Immunol., 197 (5): 1557-65 (2016)). However, prolonged activation of neutrophils can contribute to pathophysiological changes and cause acute or chronic inflammatory diseases such as chronic obstructive pulmonary disease (COPD; Lonergan et al., Respir. Res., 21 (1): 166 (2020)), cystic fibrosis (Conese et al., J. Cyst. Fibros., 2 (3): 129-35 (2003)), acute lung injury (Yang et al., Biomed. J., 44 (4): 439-46 (2020)), and acute respiratory distress syndrome bronchiectasis (Wright et al., Rheumatology (Oxford), 49 (9): 1618-31 (2010)). In addition, many studies have shown that NE also could promote tumor proliferation by degrading IRS-1 and activating the PI3K-AKT signal pathway (Ocana et al., Mol. Cancer, 16 (1): 137 (2017); and Yang et al., Mol. Med. Rep., 13 (5): 4175-82 (2016)).

### SUMMARY

This document provides methods and materials involved in binding a molecule (e.g., an antibody, an antigen binding fragment, or an antibody domain) to an NE polypeptide. For example, this document provides binders (e.g., antibodies, antigen binding fragments, and antibody domains) that bind to an NE polypeptide and methods and materials for using one or more such binders to treat a mammal (e.g., a human) having cancer and/or an inflammatory condition.

This document also provides cells (e.g., host cells) designed to express one or more binders (e.g., antibodies, antigen binding fragments, or antibody domains) having the ability to bind to an NE polypeptide and methods and materials for using such cells to treat cancer and/or an inflammatory condition.

As described herein, binders (e.g., one or more antibodies, one or more antigen binding fragments, and/or one or more antibody domains) can be designed to have the ability to bind to an NE polypeptide. For example, a binder (e.g., an antibody, an antigen binding fragment, or an antibody domain) provided herein can have the ability to bind to a polypeptide comprising, consisting essentially of, or consisting of the amino acid sequence of a human NE polypeptide as set forth in SEQ ID NO:74, SEQ ID NO:75, and/or SEQ ID NO: 166 (see, e.g., FIG. 1).

In some cases, a binder provided herein (e.g., one or more antibodies, one or more antigen binding fragments, and/or one or more antibody domains) (a) can bind to an NE polypeptide, (b) can, via the binding, inhibit the protease activity of the NE polypeptide, (c) can, via the binding, prevent the uptake of the NE polypeptide by a cell (e.g., a cancer cell), and/or (d) can, via the binding, block the ability of the NE polypeptide to promote cell differentiation (e.g., fibroblast differentiation). For example, a binder (e.g., an antibody, an antigen binding fragment, or an antibody domain) provided herein can have the ability to bind to a polypeptide comprising, consisting essentially of, or consisting of the amino acid sequence of a human NE polypeptide as set forth in SEQ ID NO:74, SEQ ID NO: 75, and/or SEQ ID NO: 166 (see, e.g., FIG. 1) in a manner that inhibits the protease activity of the polypeptide.

In some cases, two sets of three CDRs of an antigen binding fragment provided herein (e.g., SEQ ID NOs: 1-3 and 9-11) or one set of three CDRs of an antibody domain provided herein (e.g., SEQ ID NOs: 17-19) can be engineered into an antibody, antigen binding fragment, or antibody domain structure to create a binder (e.g., fully human binder) having the ability to bind to and inhibit the protease activity of an NE polypeptide.

As also described herein, binders (e.g., one or more antibodies, one or more antigen binding fragments, and/or one or more antibody domains) provided herein can be used to treat a mammal (e.g., a human) having cancer. For example, a mammal (e.g., a human) having cancer can be administered a composition comprising one or more binders (e.g., one or more antibodies, one or more antigen binding fragments, and/or one or more antibody domains) described herein to inhibit the protease activity of an NE polypeptide and/or to block the ability of cancer cells to uptake an NE polypeptide.

As also described herein, binders (e.g., one or more antibodies, one or more antigen binding fragments, and/or one or more antibody domains) provided herein can be used to treat a mammal (e.g., a human) having an inflammatory condition. For example, a mammal (e.g., a human) having an inflammatory condition can be administered a composition comprising one or more binders (e.g., one or more antibodies, one or more antigen binding fragments, and/or one or more antibody domains) described herein to inhibit the protease activity of an NE polypeptide.

As also described herein, cells (e.g., host cells) can be designed to express one or more binders (e.g., one or more antibodies, one or more antigen binding fragments, and/or one or more antibody domains) having the ability to bind to an NE polypeptide. For example, cells such as Expi 293 cells, 293F cells, or CHO cells can be engineered to express one or more antibodies, one or more antigen binding fragments, and/or one or more antibody domains having the ability to bind to an NE polypeptide and (a) inhibit the protease activity of the NE polypeptide and/or (b) block the uptake of the NE polypeptide by cells (e.g., cancer cells).

In some cases, a binder (e.g., an antibody, antigen binding fragment, and/or antibody domain) provided herein can be used to detect the presence or absence of an NE polypeptide. For example, a binder (e.g., an antibody, antigen binding fragment, and/or antibody domain) provided herein can be used to determine whether or not a sample (e.g., a biological sample) obtained from a mammal (e.g., a human) contains NE polypeptides. Having the ability to detect the presence or absence of an NE polypeptide can allow clinicians, health professionals, and patients to make better decisions about possible treatment options. For example, detection of an abundance of NE polypeptides within a mammal can allow clinicians, health professionals, and patients to select an appropriate treatment that reduces the protease activity of NE polypeptides within the mammal. Such treatments that reduce protease activity of NE polypeptides can include, without limitation, administration of anti-NE antibody, anti-NE antigen binding fragment, and/or anti-NE antibody domain provided herein.

As described herein, using a binder described herein to inhibit protease activity of and/or cell uptake of an NE polypeptide can provide an effect method for treating cancer and/or an inflammatory condition.

In general, one aspect of this document features an antibody comprising (or consisting essentially of or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO: 19 with one, two, or three amino acid additions, deletions, or substitutions), and a light chain variable domain or region. The antibody can comprise the ability to bind to SEQ ID NO:74, SEQ ID NO:75, and/or SEQ ID NO:166. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:16. The antibody can be a monoclonal antibody. The antibody can be an scFv antibody.

In another aspect, this document features an antigen binding fragment comprising (or consisting essentially of or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO: 19 with one, two, or three amino acid additions, deletions, or substitutions), and a light chain variable domain or region. The antigen binding fragment can comprise the ability to bind to SEQ ID NO: 74, SEQ ID NO:75, and/or SEQ ID NO:166. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:16. The antigen binding fragment can be monoclonal. The antigen binding fragment can be an Fab.

In another aspect, this document features an antibody domain comprising (or consisting essentially of or consisting of): (i) a heavy chain variable domain or region comprising the amino acid sequences set forth in SEQ ID NO:1 (or SEQ ID NO: 1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), or (ii) a heavy chain variable domain or region comprising the amino acid sequences set forth in SEQ ID NO: 17 (or SEQ ID NO:17 with one, two, or three amino acid additions, deletions, or substitutions), SEQ ID NO: 18 (or SEQ ID NO: 18 with one, two, or three amino acid additions, deletions, or substitutions), and SEQ ID NO: 19 (or SEQ ID NO:19 with one, two, or three amino acid additions, deletions, or substitutions). The antibody domain can comprise the ability to bind to SEQ ID NO:74, SEQ ID NO:75, and/or SEQ ID NO:166. The antibody domain can comprise the heavy chain variable domain or region of the (i). The heavy chain variable domain or region can comprise an amino acid sequence having at least 90 percent identity to the amino acid sequence set forth in SEQ ID NO:8. The antibody domain can comprise the heavy chain variable domain or region of the (ii). The heavy chain variable domain or region can comprise an amino acid sequence having at least 90 percent identity to the amino acid sequence set forth in SEQ ID NO:24. The antibody domain can be a VH domain.

In another aspect, this document features a nucleic acid comprising (or consisting essentially of or consisting of) a nucleic acid sequence encoding at least part of an antibody, an antigen-binding fragment, or antibody domain set forth an any one of the preceding three paragraphs. The nucleic acid sequence can encode the heavy chain variable domain or region of any one of (i) or (ii). The nucleic acid sequence can encode the light chain variable domain or region of any one of the (i) or (ii). The nucleic acid can be a viral vector. The nucleic acid can be a phagemid.

In another aspect, this document features a host cell comprising a nucleic acid of the preceding paragraph.

In another aspect, this document features a composition comprising (or consisting essentially of or consisting of) an antibody, an antigen binding fragment, or antibody domain described above. The composition can comprise the antibody. The composition can comprise the antigen binding fragment. The composition can comprise the antibody domain. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a composition comprising (or consisting essentially of or consisting of) a cell described above. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a method of treating a mammal having cancer or an inflammatory condition. The method comprises (or consists essentially of or consists of) administering, to the mammal, a composition of any of the two preceding paragraphs. The mammal can be a human. When treating cancer, the cancer can be selected from the group consisting of lung cancer, prostate cancer, esophageal cancer, stomach cancer, colorectal cancer, liver cancer, vaginal cancer, and cervical cancer. The number of cancer cells within the mammal can be reduced following the administering step. When treating an inflammatory condition, the inflammatory condition can be selected from the group consisting of chronic obstructive pulmonary disease (COPD), cystic fibrosis, acute lung injury, and acute respiratory distress syndrome. The level of inflammation within the mammal can be reduced following the administering step.

In another aspect, this document features a method for binding a binding molecule to an NE polypeptide. The method comprises (or consists essentially of or consists of) contacting the NE polypeptide with an antibody, an antigen binding fragment, or an antibody domain described above. The contacting can be performed in vitro. The contacting can be performed in vivo. The contacting can be performed within a mammal by administering the antibody, the antigen binding fragment, or the antibody domain to the mammal. The mammal can be a human.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts amino acid residues M1 to H267 of a human NE polypeptide (SEQ ID NO:74). The underlined and bolded amino acid sequence (residues 130 to Q247) of this human NE polypeptide depicts a mature form (SEQ ID NO:75). To identify NE polypeptide binders, S28 to H267 (SEQ ID NO:166) was linked to a pSecTag expression vector with human IgG1 Fc.

FIGS. 2A and 2B depict the amino acid sequences of the heavy chain variable domain (FIG. 2A) and the light chain variable domain (FIG. 2B) of an Fab designated Clone #1 (1C10). The CDRs, framework sequences, and constant domains of each also are provided and delineated.

FIG. 3 depicts the amino acid sequence of a VH domain designated Clone #2 (1D1.43). The CDRs and framework sequences also are provided and delineated.

FIG. 4 depicts the nucleic acid sequences encoding the indicated chains/domains of Clones #1-#2.

FIG. 5 depicts the structure of exemplary Ig molecules and provides the amino acid and nucleic acid sequences of an exemplary hinge, CH2, and CH3 regions/domains and complete Ig's.

FIG. 6 depicts the structure of exemplary scFv's.

FIGS. 7A and 7B depict the amino acid sequences of an exemplary heavy chain variable domain (FIG. 7A) and an exemplary light chain variable domain (FIG. 7B) of an exemplary scFv. The CDRs and framework sequences of each also are delineated. An exemplary linker amino acid sequence such as a linker amino acid sequence set forth in FIG. 10 can be used to link the heavy chain variable domain and the light chain variable domain together to form a scFv.

FIGS. 8A and 8B depict the amino acid sequences of an exemplary heavy chain variable domain (FIG. 8A) and an exemplary light chain variable domain (FIG. 8B) of an exemplary scFv. The CDRs and framework sequences of each also are delineated. An exemplary linker amino acid sequence such as a linker amino acid sequence set forth in FIG. 10 can be used to link the heavy chain variable domain and the light chain variable domain together to form a scFv.

FIGS. 9A and 9B depict the amino acid sequences of an exemplary heavy chain variable domain (FIG. 9A) and an exemplary light chain variable domain (FIG. 9B) of an exemplary scFv. The CDRs and framework sequences of each also are delineated. An exemplary linker amino acid sequence such as a linker amino acid sequence set forth in FIG. 10 can be used to link the heavy chain variable domain and the light chain variable domain together to form a scFv.

FIG. 10 depicts exemplary linker amino acid sequences that can be used to link a heavy chain variable domain and a light chain variable domain together to form a scFv.

FIG. 11. Binding and aggregation characterization of different format anti-hNE antibodies. (A) VH 1D1.43, Fab 1C10, VH-Fc 1D1.43, and IgG1 1C10 binding to recombinant NE measured by ELISA. Experiments were performed in duplicate. (B) Aggregation evaluation of VH 1D1, VH 1D1.43, and VH-Fc 1D1.43 measured by SEC. (C) Aggregation evaluation of Fab 1C10 and IgG1 measured by SEC. (D) Aggregation evaluation of VH and Fab measured by DLS. Antibodies were evaluated at a concentration of 1 mg/mL. (E-F) Kinetics of VH-Fc 1D1.43 (E) and IgG1 1C10 (F) binding to recombinant NE measured by BLItz. Values were reported as the mean±SD.

FIG. 12. Specificity of VH/VH-Fc and Fab/IgG1 with recombinant human NE. (A and B) Binding of Fab 1C10 (A) and VH 1D1.43 (B) to rhNE, rhMPO, and PR3 measured by ELISA. (C and D) Binding of IgG1 1C10 (C) and VH-Fc 1D1.43 (D) to rhNE, rhMPO, BSA, and PR3 measured by ELISA. rhNE, rhMPO, BSA, and PR3 were coated in 96-well microplates at 50 ng/well. Experiments were performed in duplicate. Values were reported as the mean±SD.

FIG. 13. Inhibition effects of VH/VH-Fc 1D1.43 and Fab/IgG1 1C10 on enzyme activity. (A and C) Inhibition of VH 1D1.43 (A) and VH-Fc 1D1.43 (C) on NE enzyme activity. (B and D) Inhibition of Fab 1C10 (B) and IgG1 1C10 (D) on NE enzyme activity. Each experiment were performed in duplicate. Calculation: Choose two time points (T1 and T2) in the linear range and determine the Fluorescence (FLU) at each time (FLU1 and FLU2) and use them to determine the Slope. Slope=(FLU2-FLU1)/(T2-T1)=ΔFLU/minute. % Relative inhibition=(SlopeEC-SlopeSM)/SlopeEC×100%, SlopeEC=the slope of the Sample inhibitor, and SlopeSM=the slope of the Enzyme control. Values were reported as the mean±SD.

FIG. 14. VH-Fc 1D1.43 and IgG1 1C10 inhibited NE uptaking by tumor cell. (A and B) Inhibition of VH-Fc 1D1.43 (A) and IgG1 1C10 (B) on NE uptaking by PC-3 tumor cell measured by flow cytometry. PC-3 cells were plated in a 24-well plate and incubated with serum-free medium for 24 hours, then treated with 30 nM NE-FITC with or without 100 nM antibody proteins for 1 hour. Experiments were performed in duplicate; unpaired Student's t-test, *p<0.05. Values were reported as the mean±SD.

FIG. 15. VH-Fc 1D1.43 and IgG1 1C10 inhibited NE promoted fibroblast differentiation. (A) Representative pictures of Wound healing assay. LL47 fibroblasts were plated into a 24-well plate and scratched, treated with 8 nM NE with or without antibody proteins at the different concentrations for 24 hours. (B and C) VH-Fc 1D1.43 (B) and IgG1 1C10 (C) inhibited NE-promoted LL 47 fibroblast differentiation. Experiments were performed in duplicate. Values were reported as the mean of percent wound closure±SD.

FIG. 16. Epitope mapping of VH-Fc 1D1.43 and IgG1 1C10 by using conformational epitope mappings. (A) Competition between VH-Fc 1D1.43 and IgG1 1C10 measured by BLItz. (B) The binding region of VH 1D1.43 and Fab 1C10 on hNE (PDB: 3Q76) based on the conformational epitope mapping results with the binding motifs highlighted by dots presentations. The enzymatic sites are shown by the stick models. (C and D) The detailed binding model of Fab 1C10 and VH 1D1.43 on hNE, as predicted by Z-DOCK programs. Human NE is represented as green cartoons with active site highlighted by sticks and binding epitope highlighted by blue color. Fab 1C10 and VH 1D1.43 are represented as cyan cartoons with highlighted colored CDR loops.

FIG. 17. Mapping signal of IgG1 1C10 and VH-Fc 1D1.43 binding to hNE peptide microarray. (a) Fluorescence intensity signal of IgG1 1C10 binding to hNE cyclic constrained peptides at the antibody concentration of 10 μg/mL and 30 μg/mL. (b) Fluorescence intensity signal of VH-Fc 1D1.43 binding to hNE cyclic constrained peptides at the antibody concentration of 10 μg/mL and 30 μg/mL. To perform the conformational epitope mapping, hNE were converted into cyclic constrained peptides with peptides lengths of 7, 10, and 13 amino acids and peptide-peptide overlap of 6, 9, and 12 amino acids.

### DETAILED DESCRIPTION

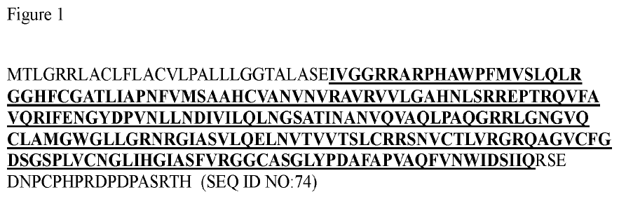

This document provides binders (e.g., antibodies, antigen binding fragments, and antibody domains) that bind (e.g., specifically bind) to an NE polypeptide (e.g., a human NE polypeptide). For example, the document provides binders (e.g., antibodies, antigen binding fragments, and antibody domains) that bind (e.g., specifically bind) to a polypeptide comprising, consisting essentially of, or consisting of the amino acid set forth in SEQ ID NO:74, SEQ ID NO:75, and/or SEQ ID NO: 166 (see, e.g., FIG. 1).

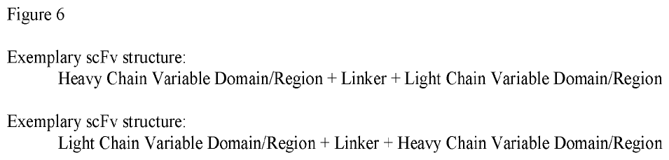

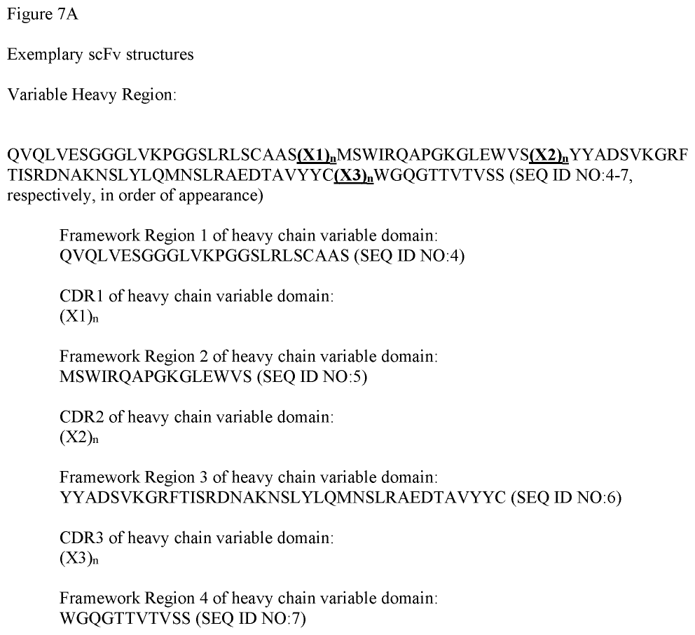

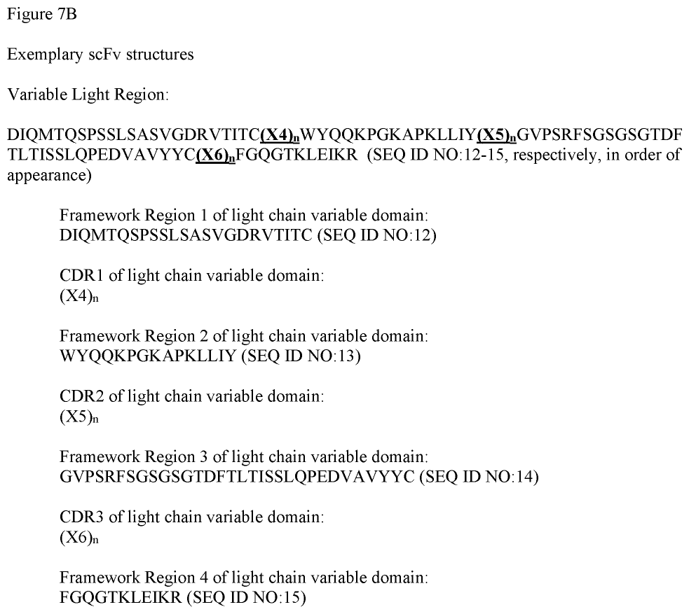

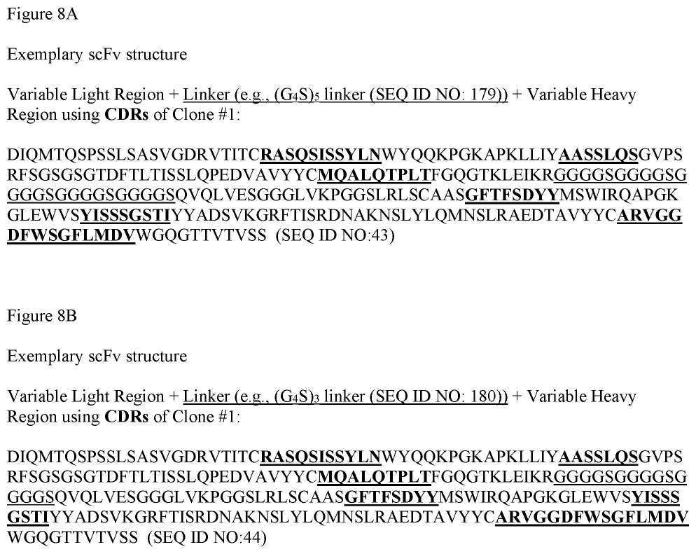

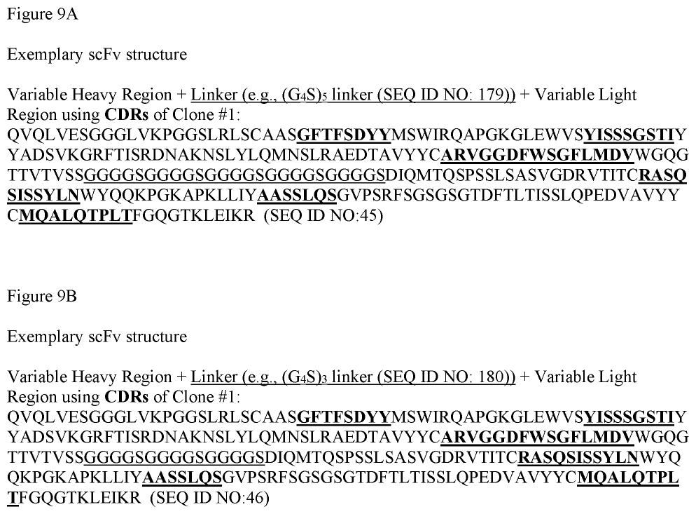

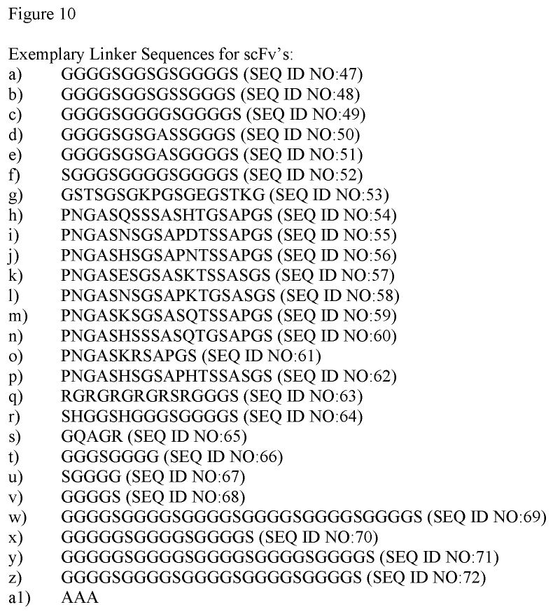

The term “antibody” as used herein includes polyclonal antibodies, monoclonal antibodies, recombinant antibodies, humanized antibodies, human antibodies, chimeric antibodies, multi-specific antibodies (e.g., bispecific antibodies) formed from at least two antibodies, diabodies, single-chain variable fragment antibodies (e.g., scFv antibodies), and tandem single-chain variable fragments antibody (e.g., taFv). A diabody can include two chains, each having a heavy chain variable domain and a light chain variable domain, either from the same or from different antibodies (see, e.g., Hornig and Färber-Schwarz, Methods Mol. Biol., 907:713-27 (2012); and Brinkmann and Kontermann, MAbs., 9 (2): 182-212 (2017)). The two variable regions can be connected by a polypeptide linker (e.g., a polypeptide linker having five to ten residues in length or a polypeptide linker as set forth in FIG. 10). In some cases, an interdomain disulfide bond can be present in one or both of the heavy chain variable domain and light chain variable domain pairs of the diabody. A scFv is a single-chain polypeptide antibody in which the heavy chain variable domain and the light chain variable domain are directly connected or connected via a polypeptide linker (e.g., a polypeptide linker having eight to 18 residues in length or a polypeptide linker as set forth in FIG. 10). See, also, Chen et al., Adv. Drug Deliv. Rev., 65 (10): 1357-1369 (2013). A scFv can be designed to have an orientation with the heavy chain variable domain being followed by the light chain variable domain or can be designed to have an orientation with the light chain variable domain being followed by the heavy chain variable domain. In both cases, the optional linker can be located between the two domains. Examples of scFv structures of scFv's provided herein include, without limitation, those structures set forth in FIGS. 6, 7A-7B, 8A-8B, and 9A-9B.

An antibody provided herein can include the CDRs as described herein (e.g., as described in Table 10) and can be configured to be a human antibody, a humanized antibody, or a chimeric antibody. In some cases, an antibody provided herein can include the CDRs as described herein (e.g., as described in Table 10) and can be a monoclonal antibody. In some cases, an antibody provided herein can include the CDRs as described herein (e.g., as described in Table 10) and can be configured as a scFv antibody.

The term “antigen binding fragment” as used herein refers to a fragment of an antibody (e.g., a fragment of a humanized antibody, a fragment of a human antibody, or a fragment of a chimeric antibody) having the ability to bind to an antigen. Examples of antigen binding fragments include, without limitation, Fab, Fab′, or F(ab′)2 antigen binding fragments. An antigen binding fragment provided herein can include the CDRs as described herein (e.g., as described in Table 10) and can be configured to be a human antigen binding fragment, a humanized antigen binding fragment, or a chimeric antigen binding fragment. In some cases, an antigen binding fragment provided herein can include the CDRs as described herein (e.g., as described in Table 10) and can be a monoclonal antigen binding fragment. In some cases, an antigen binding fragment provided herein can include the CDRs as described herein (e.g., as described in Table 10) and can be configured as an Fab antibody. In some cases, a Fab antibody can include a partial hinge sequence (e.g., SEQ ID NO:26) for disulfide bonding between heavy and light chains of the Fab.

The term “antibody domain” as used herein refers to a domain of an antibody such as a heavy chain variable domain (VH domain) or a light chain variable domain (VL domain) in the absence of one or more other domains of an antibody. In some cases, an antibody domain can be a single antibody domain (e.g., a VH domain or a VL domain) having the ability to bind to an antigen. An antibody domain provided herein can include the CDRs as described herein (e.g., as described in Table 10) and can be a human antibody domain (e.g., a human VH domain), a humanized antibody domain (e.g., a humanized VH domain), or a chimeric antibody domain (e.g., a chimeric VH domain). In some cases, an antibody domain provided herein can include the CDRs as described herein (e.g., as described in Table 10) and can be a monoclonal antibody domain. In some cases, an antibody domain provided herein can include the CDRs as described herein (e.g., as described in Table 10) and can be engineered as a single VH domain or a single VL domain.

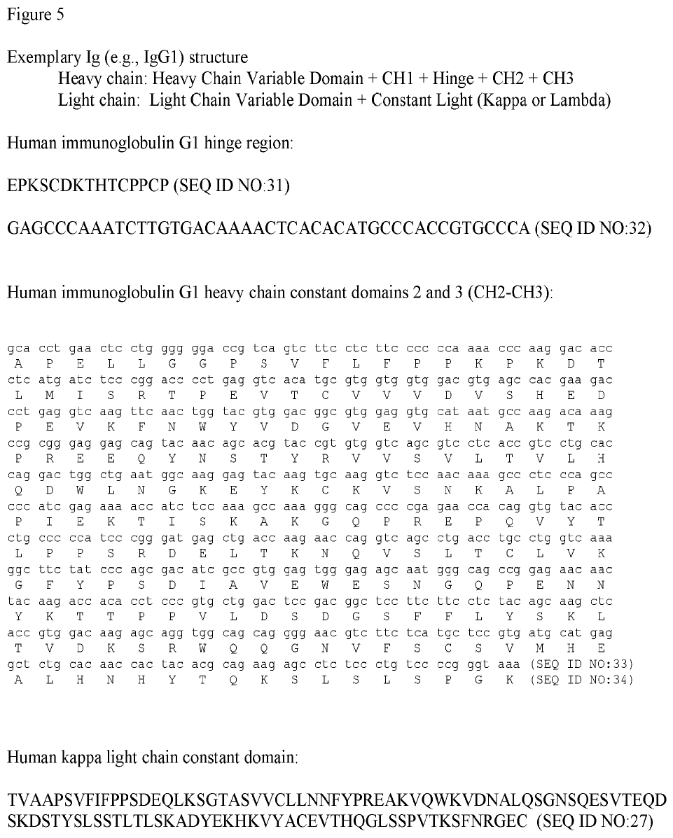

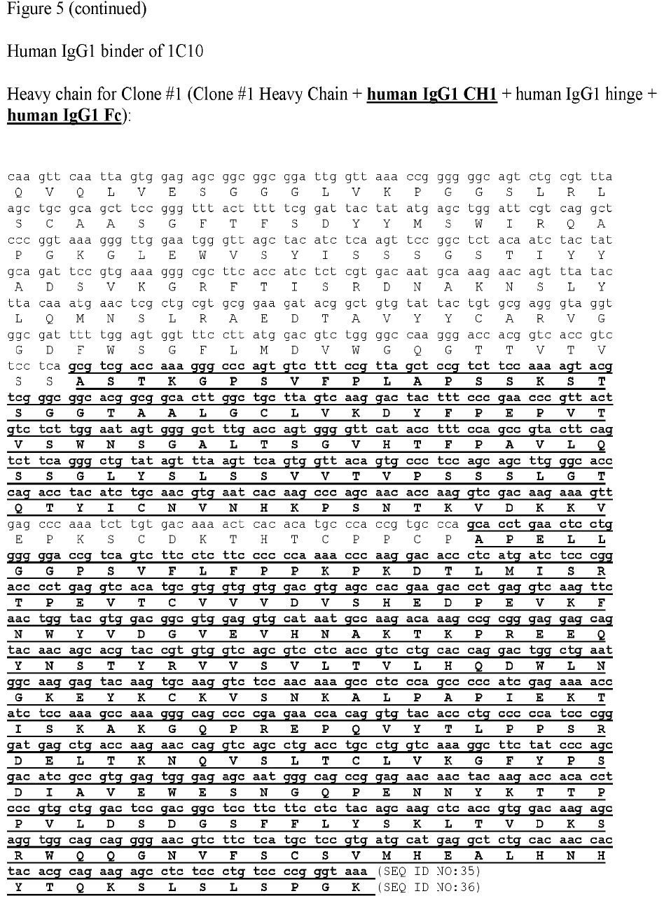

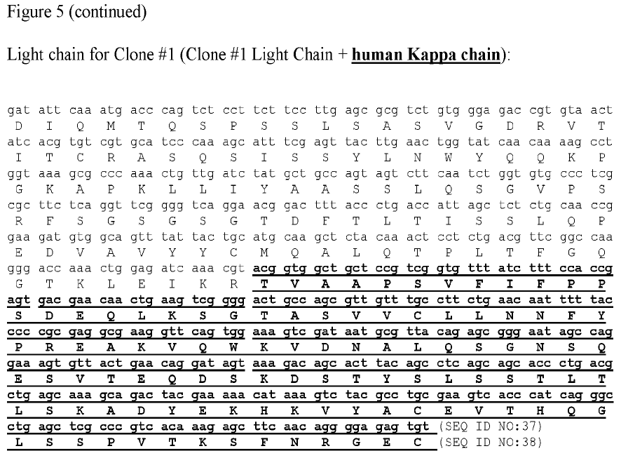

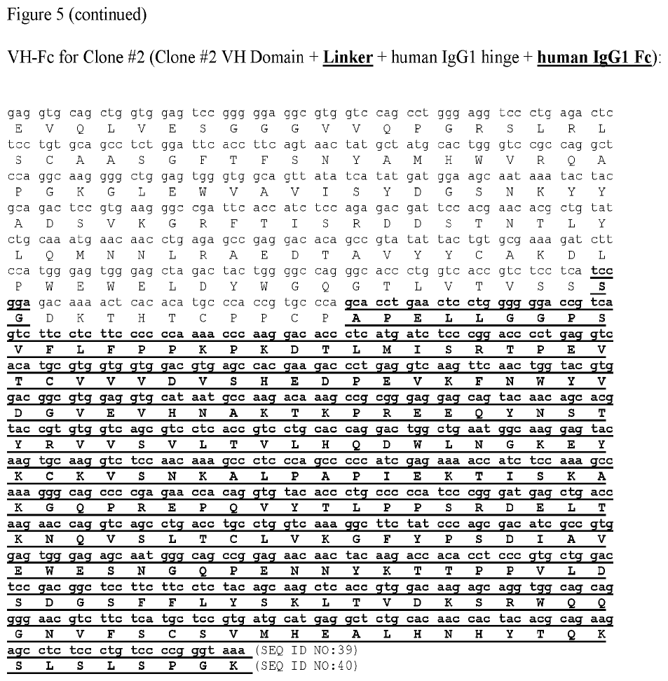

An anti-NE antibody, anti-NE antigen binding fragment, or anti-NE antibody domain provided herein can be of the IgA-, IgD-, IgE-, IgG-, or IgM-type, including IgG- or IgM-types such as, without limitation, IgG₁-, IgG₂-, IgG₃-, IgG₄-, IgM₁-, and IgM₂-types. In some cases, an antibody provided herein (e.g., an anti-NE antibody) can be a scFv antibody. In some cases, an antigen binding fragment provided herein (e.g., an anti-NE antibody fragment) can be an Fab. In some cases, an antibody provided herein (e.g., an anti-NE antibody) can be a fully intact antibody having the structure set forth in FIG. 5. In some cases, an antibody domain provided herein (e.g., an anti-NE antibody domain) can be a VH domain.

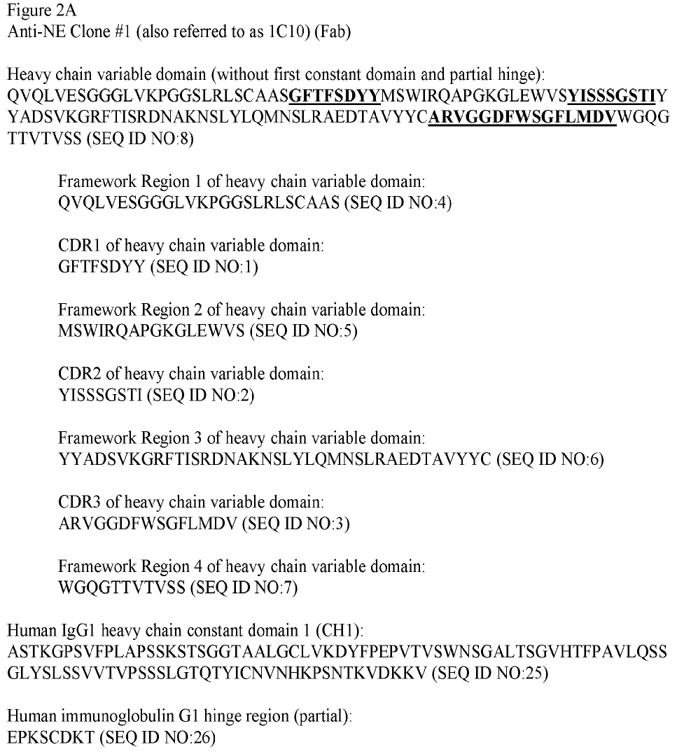

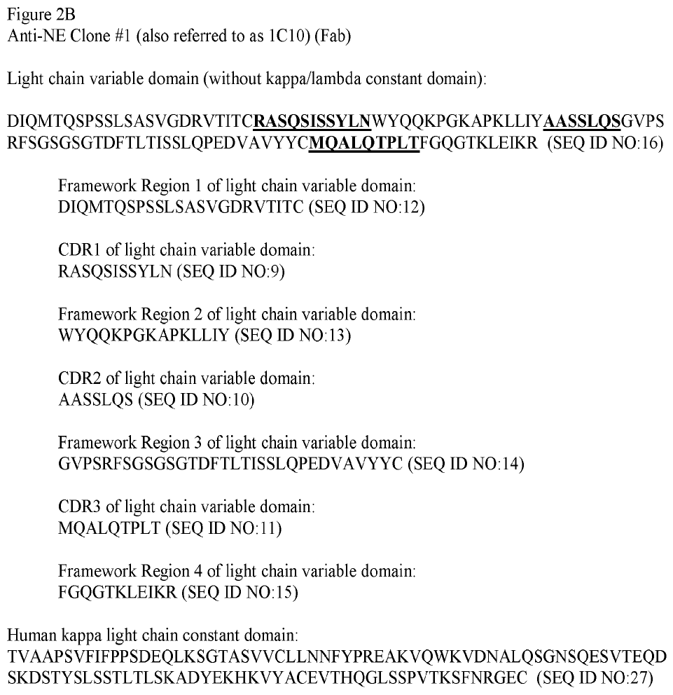

In one embodiment, a binder (e.g., an antibody, antigen binding fragment, and/or antibody domain) provided herein having the ability to bind to an NE polypeptide (e.g., a human NE polypeptide) can include (i) a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications); and/or (ii) a light chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO: 10 with one or two amino acid modifications), and a CDR3 having the amino acid sequence set forth SEQ ID NO: 11 (or a variant of SEQ ID NO:11 with one or two amino acid modifications). An example of such an antigen binding fragment having these CDRs and the ability to bind to an NE polypeptide (e.g., a human NE polypeptide) includes, without limitation, the Fab set forth in FIGS. 2A and 2B.

In some cases, a binder (e.g., an antibody, antigen binding fragment, and/or antibody domain) provided herein having the ability to bind to an NE polypeptide (e.g., a human NE polypeptide) and (a) a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications) and/or (b) a light chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO: 10 with one or two amino acid modifications), and a CDR3 having the amino acid sequence set forth SEQ ID NO:11 (or a variant of SEQ ID NO:11 with one or two amino acid modifications) can include any appropriate framework regions. For example, such a binder (e.g., an antibody, antigen binding fragment, and/or antibody domain) can include (a) a heavy chain variable domain that includes a framework region 1 having the amino acid sequence set forth in SEQ ID NO:4 (or a variant of SEQ ID NO:4 with one, two, three, four, five, six, seven, eight, nine, ten, or more amino acid modifications), a framework region 2 having the amino acid sequence set forth in SEQ ID NO:5 (or a variant of SEQ ID NO:5 with one, two, three, four, five, six, seven, eight, nine, ten, or more amino acid modifications), a framework region 3 having the amino acid sequence set forth in SEQ ID NO:6 (or a variant of SEQ ID NO:6 with one, two, three, four, five, six, seven, eight, nine, ten, or more amino acid modifications), and a framework region 4 having the amino acid sequence set forth in SEQ ID NO:7 (or a variant of SEQ ID NO:7 with one, two, three, four, five, six, seven, eight, nine, ten, or more amino acid modifications) and/or (b) a light chain variable domain that includes a framework region 1 having the amino acid sequence set forth in SEQ ID NO:12 (or a variant of SEQ ID NO: 12 with one, two, three, four, five, six, seven, eight, nine, ten, or more amino acid modifications), a framework region 2 having the amino acid sequence set forth in SEQ ID NO: 13 (or a variant of SEQ ID NO:13 with one, two, three, four, five, six, seven, eight, nine, ten, or more amino acid modifications), a framework region 3 having the amino acid sequence set forth in SEQ ID NO:14 (or a variant of SEQ ID NO: 14 with one, two, three, four, five, six, seven, eight, nine, ten, or more amino acid modifications), and a framework region 4 having the amino acid sequence set forth in SEQ ID NO:15 (or a variant of SEQ ID NO: 15 with one, two, three, four, five, six, seven, eight, nine, ten, or more amino acid modifications).

In some cases, a binder (e.g., an antibody, antigen binding fragment, and/or antibody domain) having any of the CDRs set forth in FIGS. 2A or 2B can be designed to include framework regions as set forth in FIGS. 2A and 2B or can be designed to include one or more framework regions from another antibody, antibody fragment, or antibody domain. For example, an Fab can be designed to include the six CDRs set forth in FIGS. 2A and 2B and the framework regions set forth in FIGS. 2A and 2B except that framework region 1 having the amino acid set forth in SEQ ID NO:4 is replaced with a framework region 1 having a different amino acid sequence. In some cases, a scFv can be designed to include the six CDRs set forth in FIGS. 2A and 2B and the framework regions set forth in FIGS. 2A and 2B. In some cases, a scFv can be designed to include the six CDRs set forth in FIGS. 2A and 2B and the framework regions set forth in FIGS. 2A and 2B except that framework region 1 having the amino acid set forth in SEQ ID NO:4 is replaced with a framework region 1 having a different amino acid sequence.

In some cases, a binder (e.g., an antibody, antigen binding fragment, and/or antibody domain) provided herein having the ability to bind to an NE polypeptide (e.g., a human NE polypeptide) can include (a) a heavy chain variable domain that includes an amino acid sequence having at least 90 percent identity to the amino acid sequence set forth in SEQ ID NO:8 and/or (b) a light chain variable domain that includes an amino acid sequence having at least 90 percent identity to the amino acid sequence set forth in SEQ ID NO:16. For example, a binder (e.g., an antibody, antigen binding fragment, and/or antibody domain) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8 and/or (b) a light chain variable domain that includes an amino acid sequence having at least 90, 91, 92, 93, 94, 95, 96, 97, 98, or 99 percent identity to the amino acid sequence set forth in SEQ ID NO:16. In some cases, a binder (e.g., an antibody, antigen binding fragment, and/or antibody domain) provided herein can include (a) a heavy chain variable domain that includes an amino acid sequence having 100 percent identity to the amino acid sequence set forth in SEQ ID NO:8 and/or (b) a light chain variable domain that includes an amino acid sequence having 100 percent identity to the amino acid sequence set forth in SEQ ID NO:16.

In some cases, a binder (e.g., an antibody, antigen binding fragment, and/or antibody domain) provided herein having the ability to bind to an NE polypeptide (e.g., a human NE polypeptide) can include (a) a heavy chain variable domain that includes an amino acid sequence having at least 90 percent identity to the amino acid sequence set forth in SEQ ID NO:8, provided that the heavy chain variable domain includes the amino acid sequences set forth in SEQ ID NOs: 1, 2, and 3, and/or (b) a light chain variable domain that includes an amino acid sequence having at least 90 percent identity to the amino acid sequence set forth in SEQ ID NO: 16, provided that the light chain variable domain includes the amino acid sequences set forth in SEQ ID NOs: 9, 10, and 11. For example, a binder (e.g., an antibody, antigen binding fragment, and/or antibody domain) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8, provided that the heavy chain variable domain includes the amino acid sequences set forth in SEQ ID NOs: 1, 2, and 3, and/or (b) a light chain variable domain that includes an amino acid sequence having at least 90, 91, 92, 93, 94, 95, 96, 97, 98, or 99 percent identity to the amino acid sequence set forth in SEQ ID NO: 16, provided that the light chain variable domain includes the amino acid sequences set forth in SEQ ID NOs: 9, 10, and 11.

In some cases, a binder (e.g., an antibody, antigen binding fragment, and/or antibody domain) provided herein having the ability to bind to an NE polypeptide (e.g., a human NE polypeptide) can include (a) a heavy chain variable domain having the amino acid sequence set forth in SEQ ID NO:8 or the amino acid set forth in SEQ ID NO:8 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO: 16 or the amino acid set forth in SEQ ID NO:16 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to an NE polypeptide (e.g., a human NE polypeptide), can include a heavy chain variable domain having the amino acid sequence set forth in SEQ ID NO:8 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 1, 2, and 3, and can include a light chain variable domain having the amino acid sequence set forth in SEQ ID NO: 16 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs: 9, 10, and 11.

In some cases, a binder (e.g., an antibody, antigen binding fragment, and/or antibody domain) provided herein having the ability to bind to an NE polypeptide (e.g., a human NE polypeptide) can include (a) a heavy chain variable domain comprising (i) a CDR1 that comprises, consists essentially of, or consists of the amino acid sequence set forth in SEQ ID NO:1, (ii) a CDR2 that comprises, consists essentially of, or consists of the amino acid sequence set forth in SEQ ID NO:2, and (iii) a CDR3 that comprises, consists essentially of, or consists of the amino acid sequence set forth in SEQ ID NO:3, and/or (b) a light chain variable domain comprising (i) a CDR1 that comprises, consists essentially of, or consists of the amino acid sequence set forth in SEQ ID NO:9, (ii) a CDR2 that comprises, consists essentially of, or consists of the amino acid sequence set forth in SEQ ID NO:10, and (iii) a CDR3 that comprises, consists essentially of, or consists of the amino acid sequence set forth in SEQ ID NO:11. As used herein, a “CDR1 that consists essentially of the amino acid sequence set forth in SEQ ID NO:1” is a CDR1 that has zero, one, or two amino acid substitutions within SEQ ID NO:1, that has zero, one, two, three, four, or five amino acid residues directly preceding SEQ ID NO:1, and/or that has zero, one, two, three, four, or five amino acid residues directly following SEQ ID NO:1, provided that the binder (e.g., an antibody, antigen binding fragment, and/or antibody domain) maintains its basic ability to bind to an NE polypeptide (e.g., a human NE polypeptide). Examples of a CDR1 that consists essentially of the amino acid sequence set forth in SEQ ID NO: 1 include, without limitation, those set forth in Table 1.

TABLE 1
Exemplary CDR1s that consist essentially of the
amino acid sequence set forth in SEQ ID NO: 1.

| Sequence | SEQ ID NO: |
|---|---|
| GFTFSSYY | 76 |
| GFTFSNHY | 77 |
| GYTFTDYY | 78 |
| GFTFNDHY | 79 |
| GFTFSDYF | 80 |
| GFTFSEYY | 81 |
| GFTFTDYY | 82 |
| GFTFGDYY | 83 |
| GFTFADYY | 84 |
| GFSFSDYY | 85 |

As used herein, a “CDR2 that consists essentially of the amino acid sequence set forth in SEQ ID NO:2” is a CDR2 that has zero, one, or two amino acid substitutions within SEQ ID NO:2, that has zero, one, two, three, four, or five amino acid residues directly preceding SEQ ID NO:2, and/or that has zero, one, two, three, four, or five amino acid residues directly following SEQ ID NO:2, provided that the binder (e.g., an antibody, antigen binding fragment, and/or antibody domain) maintains its basic ability to bind to an NE polypeptide (e.g., a human NE polypeptide). Examples of a CDR2 that consists essentially of the amino acid sequence set forth in SEQ ID NO:2 include, without limitation, those set forth in Table 2.

TABLE 2
Exemplary CDR2s that consist essentially of the
amino acid sequence set forth in SEQ ID NO: 2.

| Sequence | SEQ ID NO: |
|---|---|
| YISSSGNTI | 86 |
| YISNSGSTI | 87 |
| YVSSSGSTI | 88 |
| YISSSGTTI | 89 |
| YIASSGSTI | 90 |
| YISSTGSTI | 91 |
| YISSSGSAI | 92 |
| YINSSGSTI | 93 |
| YISSSSSTI | 94 |
| YIGSSGSTI | 95 |

As used herein, a “CDR3 that consists essentially of the amino acid sequence set forth in SEQ ID NO:3” is a CDR3 that has zero, one, or two amino acid substitutions within SEQ ID NO:3, that has zero, one, two, three, four, or five amino acid residues directly preceding SEQ ID NO:3, and/or that has zero, one, two, three, four, or five amino acid residues directly following SEQ ID NO:3, provided that the binder (e.g., an antibody, antigen binding fragment, and/or antibody domain) maintains its basic ability to bind to an NE polypeptide (e.g., a human NE polypeptide). Examples of a CDR3 that consists essentially of the amino acid sequence set forth in SEQ ID NO:3 include, without limitation, those set forth in Table 3.

TABLE 3
Exemplary CDR3s that consist essentially of the
amino acid sequence set forth in SEQ ID NO: 3.

| Sequence | SEQ ID NO: |
|---|---|
| ARVGGDFWSGYLMDV | 96 |
| ARVGGDFWSGYFMDV | 97 |
| ARVGSDFWSGFRMDV | 98 |
| AREGGDFWSGFLMDV | 99 |
| ARGGNDFWSGYLMDV | 100 |
| ARVGNDFWSGYWMDV | 101 |
| ARTEGDFWSGYLMDV | 102 |
| ARGGGDFWSGYMMDV | 103 |
| ARMGDDFWSGYLMDV | 104 |
| ARGGGDFWRGYYMDV | 105 |

As used herein, a “CDR1 that consists essentially of the amino acid sequence set forth in SEQ ID NO:9” is a CDR1 that has zero, one, or two amino acid substitutions within SEQ ID NO:9, that has zero, one, two, three, four, or five amino acid residues directly preceding SEQ ID NO:9, and/or that has zero, one, two, three, four, or five amino acid residues directly following SEQ ID NO:9, provided that the binder (e.g., an antibody, antigen binding fragment, and/or antibody domain) maintains its basic ability to bind to an NE polypeptide (e.g., a human NE polypeptide). Examples of a CDR1 that consists essentially of the amino acid sequence set forth in SEQ ID NO:9 include, without limitation, those set forth in Table 4.

TABLE 4
Exemplary CDR1s that consist essentially of the
amino acid sequence set forth in SEQ ID NO: 9.

| Sequence | SEQ ID NO: |
|---|---|
| RASQSVSSYLN | 106 |
| RASQSISTYLN | 107 |
| RASQSISNYLN | 108 |
| RASQSITSYLN | 109 |
| RASQSISAYLN | 110 |
| RASQSINSYLN | 111 |
| RASQTISSYLN | 112 |
| RSSQSISSYLN | 113 |
| RASQSISSYLD | 114 |
| RASQSIASYLN | 115 |

As used herein, a “CDR2 that consists essentially of the amino acid sequence set forth in SEQ ID NO:10” is a CDR2 that has zero, one, or two amino acid substitutions within SEQ ID NO:10, that has zero, one, two, three, four, or five amino acid residues directly preceding SEQ ID NO:10, and/or that has zero, one, two, three, four, or five amino acid residues directly following SEQ ID NO:10, provided that the binder (e.g., an antibody, antigen binding fragment, and/or antibody domain) maintains its basic ability to bind to an NE polypeptide (e.g., a human NE polypeptide). Examples of a CDR2 that consists essentially of the amino acid sequence set forth in SEQ ID NO:10 include, without limitation, those set forth in Table 5.

TABLE 5
Exemplary CDR2s that consist essentially of the
amino acid sequence set forth in SEQ ID NO: 10.

| Sequence | SEQ ID NO: |
|---|---|
| AAASLQS | 116 |
| AASALQS | 117 |
| AASTLQS | 118 |
| AASNLQS | 119 |
| ASSSLQS | 120 |
| AANSLQS | 121 |
| AASSMQS | 122 |
| AANSMQS | 123 |
| AAASMQS | 124 |
| ASSTLQS | 125 |

As used herein, a “CDR3 that consists essentially of the amino acid sequence set forth in SEQ ID NO:11” is a CDR3 that has zero, one, or two amino acid substitutions within SEQ ID NO:11, that has zero, one, two, three, four, or five amino acid residues directly preceding SEQ ID NO:11, and/or that has zero, one, two, three, four, or five amino acid residues directly following SEQ ID NO:11, provided that the binder (e.g., an antibody, antigen binding fragment, and/or antibody domain) maintains its basic ability to bind to an NE polypeptide (e.g., a human NE polypeptide). Examples of a CDR3 that consists essentially of the amino acid sequence set forth in SEQ ID NO:11 include, without limitation, those set forth in Table 6.

TABLE 6
Exemplary CDR3s that consist essentially of the
amino acid sequence set forth in SEQ ID NO: 11.

| Sequence | SEQ ID NO: |
|---|---|
| MQALQSPLT | 126 |
| MQALHTPLT | 127 |
| MQTLQTPLT | 128 |
| MQALETPLT | 129 |
| MQALQAPLT | 130 |
| MQALQNPLT | 131 |
| MQAIQTPLT | 132 |
| MQVLQTPLT | 133 |
| MQALRTPLT | 134 |
| MQALQTQLT | 135 |

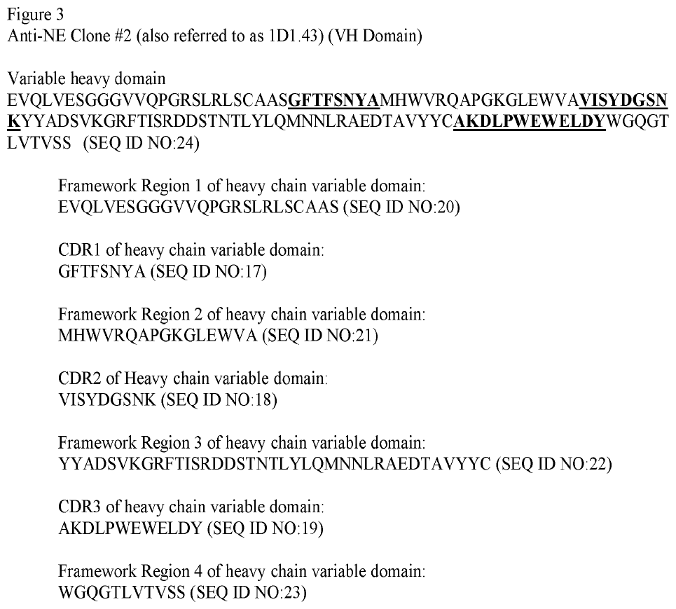

In another embodiment, a binder (e.g., an antibody, antigen binding fragment, and/or antibody domain) provided herein having the ability to bind to an NE polypeptide (e.g., a human NE polypeptide) can include a variable heavy domain having a CDR1 having the amino acid sequence set forth in SEQ ID NO: 17 (or a variant of SEQ ID NO: 17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO: 19 (or a variant of SEQ ID NO: 19 with one or two amino acid modifications). An example of such a variable heavy domain having these CDRs and the ability to bind to an NE polypeptide (e.g., a human NE polypeptide) includes, without limitation, the VH domain set forth in FIG. 3.

In some cases, a binder (e.g., an antibody, antigen binding fragment, and/or antibody domain) provided herein having the ability to bind to an NE polypeptide (e.g., a human NE polypeptide) and having a variable heavy domain having a CDR1 having the amino acid sequence set forth in SEQ ID NO:17 (or a variant of SEQ ID NO: 17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO: 19 with one or two amino acid modifications) can include any appropriate framework regions. For example, such a binder (e.g., an antibody, antigen binding fragment, and/or antibody domain) can include a variable heavy domain that includes a framework region 1 having the amino acid sequence set forth in SEQ ID NO: 20 (or a variant of SEQ ID NO:20 with one, two, three, four, five, six, seven, eight, nine, ten, or more amino acid modifications), a framework region 2 having the amino acid sequence set forth in SEQ ID NO:21 (or a variant of SEQ ID NO:21 with one, two, three, four, five, six, seven, eight, nine, ten, or more amino acid modifications), a framework region 3 having the amino acid sequence set forth in SEQ ID NO:22 (or a variant of SEQ ID NO:22 with one, two, three, four, five, six, seven, eight, nine, ten, or more amino acid modifications), and a framework region 4 having the amino acid sequence set forth in SEQ ID NO:23 (or a variant of SEQ ID NO:23 with one, two, three, four, five, six, seven, eight, nine, ten, or more amino acid modifications).

In some cases, a binder (e.g., an antibody, antigen binding fragment, and/or antibody domain) having any of the CDRs set forth in FIG. 3 can be designed to include framework regions as set forth in FIG. 3 or can be designed to include one or more framework regions from another antibody or antibody fragment. For example, a VH domain can be designed to include the three CDRs set forth in FIG. 3 and the framework regions set forth in FIG. 3 except that framework region 1 having the amino acid set forth in SEQ ID NO:20 is replaced with a framework region 1 having a different amino acid sequence.

In some cases, a binder (e.g., an antibody, antigen binding fragment, and/or antibody domain) provided herein having the ability to bind to an NE polypeptide (e.g., a human NE polypeptide) can include a variable heavy domain that includes an amino acid sequence having at least 90 percent identity to the amino acid sequence set forth in SEQ ID NO: 24. For example, a binder (e.g., an antibody, antigen binding fragment, and/or antibody domain) provided herein can include a variable heavy domain that includes an amino acid sequence having at least 90, 91, 92, 93, 94, 95, 96, 97, 98, or 99 percent identity to the amino acid sequence set forth in SEQ ID NO:24. In some cases, a binder (e.g., an antibody, antigen binding fragment, and/or antibody domain) provided herein can include a variable heavy domain that includes an amino acid sequence having 100 percent identity to the amino acid sequence set forth in SEQ ID NO:24.

In some cases, a binder (e.g., an antibody, antigen binding fragment, and/or antibody domain) provided herein having the ability to bind to an NE polypeptide (e.g., a human NE polypeptide) can include a variable heavy domain that includes an amino acid sequence having at least 90 percent identity to the amino acid sequence set forth in SEQ ID NO: 24, provided that the heavy chain variable domain includes the amino acid sequences set forth in SEQ ID NOs: 17, 18, and 19. For example, a binder (e.g., an antibody, antigen binding fragment, and/or antibody domain) provided herein can include a variable heavy domain that includes an amino acid sequence having at least 90, 91, 92, 93, 94, 95, 96, 97, 98, or 99 percent identity to the amino acid sequence set forth in SEQ ID NO: 24, provided that the heavy chain variable domain includes the amino acid sequences set forth in SEQ ID NOs: 17, 18, and 19.

In some cases, a binder (e.g., an antibody, antigen binding fragment, and/or antibody domain) provided herein having the ability to bind to an NE polypeptide (e.g., a human NE polypeptide) can include a variable heavy domain having the amino acid sequence set forth in SEQ ID NO:24 or the amino acid set forth in SEQ ID NO:24 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to an NE polypeptide (e.g., a human NE polypeptide), can include a variable heavy domain having the amino acid sequence set forth in SEQ ID NO:24 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 17, 18, and 19.

In some cases, a binder (e.g., an antibody, antigen binding fragment, and/or antibody domain) provided herein having the ability to bind to an NE polypeptide (e.g., a human NE polypeptide) can include a variable heavy domain comprising (i) a CDR1 that comprises, consists essentially of, or consists of the amino acid sequence set forth in SEQ ID NO: 17, (ii) a CDR2 that comprises, consists essentially of, or consists of the amino acid sequence set forth in SEQ ID NO: 18, and (iii) a CDR3 that comprises, consists essentially of, or consists of the amino acid sequence set forth in SEQ ID NO: 19. As used herein, a “CDR1 that consists essentially of the amino acid sequence set forth in SEQ ID NO:17” is a CDR1 that has zero, one, or two amino acid substitutions within SEQ ID NO: 17, that has zero, one, two, three, four, or five amino acid residues directly preceding SEQ ID NO:17, and/or that has zero, one, two, three, four, or five amino acid residues directly following SEQ ID NO: 17, provided that the binder (e.g., an antibody, antigen binding fragment, and/or antibody domain) maintains its basic ability to bind to an NE polypeptide (e.g., a human NE polypeptide). Examples of a CDR1 that consists essentially of the amino acid sequence set forth in SEQ ID NO:17 include, without limitation, those set forth in Table 7.

TABLE 7
Exemplary CDR1s that consist essentially of the
amino acid sequence set forth in SEQ ID NO: 17.

| Sequence | SEQ ID NO: |
|---|---|
| GFSFSNYA | 136 |
| GFTFANYA | 137 |
| GFTFNNYA | 138 |
| GFTFSNSA | 139 |
| GFTFTNYA | 140 |
| GFTFSDYA | 141 |
| GFTYSNYA | 142 |
| GYTFSNYA | 143 |
| GFTFGNYA | 144 |
| GFTFSHYA | 145 |

As used herein, a “CDR2 that consists essentially of the amino acid sequence set forth in SEQ ID NO:18” is a CDR2 that has zero, one, or two amino acid substitutions within SEQ ID NO:18, that has zero, one, two, three, four, or five amino acid residues directly preceding SEQ ID NO: 18, and/or that has zero, one, two, three, four, or five amino acid residues directly following SEQ ID NO:18, provided that the binder (e.g., an antibody, antigen binding fragment, and/or antibody domain) maintains its basic ability to bind to an NE polypeptide (e.g., a human NE polypeptide). Examples of a CDR2 that consists essentially of the amino acid sequence set forth in SEQ ID NO:18 include, without limitation, those set forth in Table 8.

TABLE 8
Exemplary CDR2s that consist essentially of the
amino acid sequence set forthin SEQ ID NO: 18.

| Sequence | SEQ ID NO: |
|---|---|
| VISFDGSNK | 146 |
| VISYNGSNK | 147 |
| VVSYDGSNK | 148 |
| VISYDGTNK | 149 |
| VIAYDGSNK | 150 |
| VISYDGSDK | 151 |
| VISYEGSNK | 152 |
| VISYDGNNK | 153 |
| VISYDGANK | 154 |
| VITYDGSNK | 155 |

As used herein, a “CDR3 that consists essentially of the amino acid sequence set forth in SEQ ID NO:19” is a CDR3 that has zero, one, or two amino acid substitutions within SEQ ID NO:19, that has zero, one, two, three, four, or five amino acid residues directly preceding SEQ ID NO:19, and/or that has zero, one, two, three, four, or five amino acid residues directly following SEQ ID NO:19, provided that the binder (e.g., an antibody, antigen binding fragment, and/or antibody domain) maintains its basic ability to bind to an NE polypeptide (e.g., a human NE polypeptide). Examples of a CDR3 that consists essentially of the amino acid sequence set forth in SEQ ID NO:19 include, without limitation, those set forth in Table 9.

TABLE 9
Exemplary CDR3s that consist essentially of the
amino acid sequence set forth in SEQ ID NO: 19.

| Sequence | SEQ ID NO: |
|---|---|
| AKILPWEKELDY | 156 |
| AEDLPWSWDLDY | 157 |
| AKDLSWNWELDF | 158 |
| AKALAWEWELEY | 159 |
| AKNLPWEWELDY | 160 |
| AKDPPWGWELDY | 161 |
| AKKLPWEWELDY | 162 |
| AMDLSWDWALNY | 163 |
| AKELPWQWEEDY | 164 |
| AKDLSWQWELEF | 165 |

When designing a single chain antibody (e.g., a scFv) having a heavy chain variable domain and a light chain variable domain, the two regions can be directly connected or can be connected using any appropriate linker sequence. For example, a heavy chain variable domain having the CDRs of SEQ ID NOs: 1-3 or SEQ ID NOs: 17-19 can be directly connected to a light chain variable domain having the CDRs of SEQ ID NOs: 9-11 or SEQ ID NOs: 25-27, respectively, via a linker sequence. Examples of linker sequences that can be used to connect a heavy chain variable domain and a light chain variable domain to create a scFv include, without limitation, those linkers set forth in FIG. 10.

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a binder (e.g., an antibody, antigen binding fragment, and/or antibody domain) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr. Opin. Biotechnol. 3:348-354 (1992). Both naturally occurring and non-naturally occurring amino acids (e.g., artificially-derivatized amino acids) can be used to generate an amino acid sequence variant provided herein.

A representative number of binders (e.g., antibodies, antigen binding fragments, and/or antibody domains) having the ability to bind to an NE polypeptide (e.g., a human NE polypeptide) are further described in Table 10.

TABLE 10
Representative number of binders.

| | | SEQ ID NOs of | | | SEQ ID NOs of | |
|---|---|---|---|---|---|---|
| | SEQ ID NOs of | Heavy Chain | | SEQ ID NOs of | Light Chain | |
| | Heavy Chain | Variable | SEQ ID NO of | Light Chain | Variable | SEQ ID NO of |
| Clone # | Variable | Domain/Region | Heavy Chain | Variable | Domain/Region | Light Chain |
| (Antibody | Domain/Region | Framework | Variable | Domain/Region | Framework | Variable |
| type) | CDRs | Regions | Domain/Region | CDRs | Regions | Domain/Region |
| #1 (Fab) | 1, 2, 3 | 4, 5, 6, 7 | 8 | 9, 10, 11 | 12, 13, 14, 15 | 16 |
| #2 (VH) | 17, 18, 19 | 20, 21, 22, 23 | 24 | | | |

Table 11 includes an alternative designation that can be used to refer to each of Clones #1-#2.

TABLE 11
Alternative nomenclature for Clones #1-#2.

| Clone # | Alternative names |
|---|---|
| 1 | 1C10 |
| 2 | 1D1.43 |

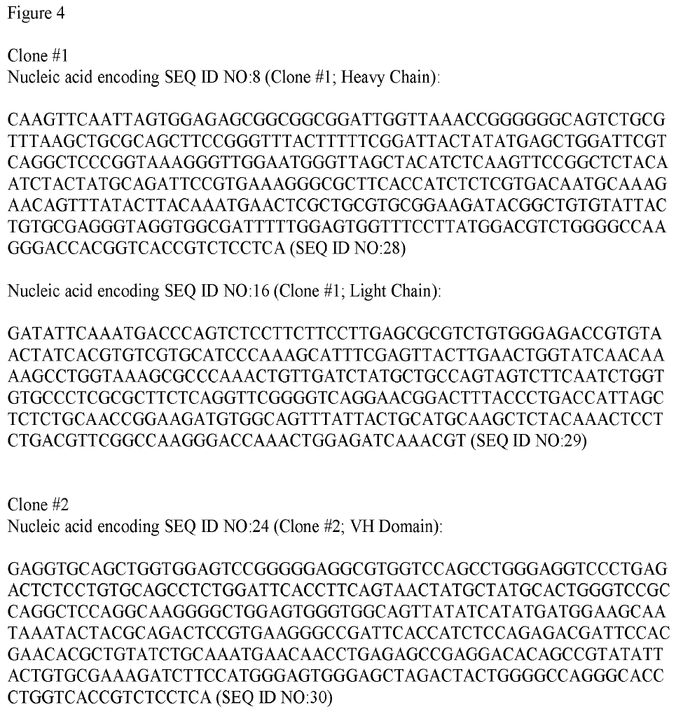

The binders (e.g., antibodies, antigen binding fragments, and/or antibody domains) provided herein can be produced using any appropriate method. For example, the binders (e.g., antibodies, antigen binding fragments, and/or antibody domains) provided herein can be produced in recombinant host cells. For example, a nucleic acid encoding a binder (e.g., an antibody, antigen binding fragment, and/or antibody domain) provided herein can be constructed, introduced into an expression vector, and expressed in suitable host cells. FIG. 4 is a sequence listing of nucleic acid sequences encoding exemplary binders (e.g., antibodies, antigen binding fragments, and/or antibody domains) described herein. In some cases, a binder (e.g., an antibody, antigen binding fragment, and/or antibody domain) provided herein can be recombinantly produced in prokaryotic hosts such as E. coli, Bacillus brevis, Bacillus subtilis, Bacillus megaterium, Lactobacillus zeae casei, or Lactobacillus paracasei. A binder (e.g., an antibody, antigen binding fragment, and/or antibody domain) provided herein also can be recombinantly produced in eukaryotic hosts such as yeast (e.g., Pichia pastoris, Saccharomyces cerevisiae, Hansenula polymorpha, Schizosaccharomyces pombe, Schwanniomyces occidentalis, Kluyveromyces lactis, or Yarrowia lipolytica), filamentous fungi of the genera Trichoderma (e.g., T. reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NS0 cells, baby hamster kidney (BHK) cells, or human embryonic kidney cell line HEK293). See, for example, the Frenzel et al. reference (Front Immunol., 4:217 (2013)).

In some cases, an antigen binding fragment or antibody domain provided herein can be produced by proteolytic digestion of an intact antibody. For example, an antigen binding fragment can be obtained by treating an antibody with an enzyme such as papain or pepsin. Papain digestion of whole antibodies can be used to produce F(ab)₂ or Fab fragments, while pepsin digestion of whole antibodies can be used to produce F(ab′)₂ or Fab′ fragments.

In some cases, a binder (e.g., an antibody, antigen binding fragment, and/or antibody domain) provided herein can be substantially pure. The term “substantially pure” as used herein with reference to a binder (e.g., an antibody, antigen binding fragment, and/or antibody domain) refers to the binder (e.g., an antibody, antigen binding fragment, and/or antibody domain) as being substantially free of other polypeptides, lipids, carbohydrates, and nucleic acid with which it is naturally associated. Thus, a substantially pure binder (e.g., an antibody, antigen binding fragment, and/or antibody domain) provided herein is any binder (e.g., an antibody, antigen binding fragment, and/or antibody domain) that is removed from its natural environment and is at least 60 percent pure. A substantially pure binder (e.g., an antibody, antigen binding fragment, and/or antibody domain) provided herein can be at least about 65, 70, 75, 80, 85, 90, 95, or 99 percent pure.

This document also provides bispecific binders (e.g., bispecific antibodies, bispecific antigen binding fragments, and/or bispecific antibody domains) that bind to two different epitopes with at least one being an epitope of an NE polypeptide (e.g., a human NE polypeptide). In some cases, a bispecific binder provided herein can be designed to bind to two different epitopes of the same NE polypeptide (e.g., a human NE polypeptide). In some cases, a bispecific binder provided herein can bind to an NE polypeptide (e.g., a human NE polypeptide) and to an epitope on a different polypeptide (e.g., a CD3 polypeptide). Bispecific binders can be produced by chemically conjugating two different binders (e.g., antibodies, antigen binding fragments, and/or antibody domains) together. Bispecific binders also can be produced by fusing two antibody-producing cells, e.g., hybridomas, to make a hybrid cell line that produces two different heavy and two different light chains within the same cell, which can result in, for example, bispecific IgG molecules. See, Brinkmann and Kontermann, MAbs., 9 (2): 182-212 (2017).

In some cases, a binder (e.g., an antibody, antigen binding fragment, and/or antibody domain) provided herein can be fused or conjugated (e.g., covalently or non-covalently attached) to another polypeptide or other moiety to provide a fusion protein or conjugate. For example, a binder (e.g., an antibody, antigen binding fragment, and/or antibody domain) provided herein can be conjugated (e.g., covalently or non-covalently attached) to a polymer (e.g., polyethylene glycol (PEG), polyethylenimine (PEI) modified with PEG (PEI-PEG), and/or polyglutamic acid (PGA) (N-(2-Hydroxypropyl) methacrylamide (HPMA) copolymers), hyaluronic acid, a fluorescent substance, a luminescent substance, a hapten, an enzyme, a metal chelate, a drug, a radioisotope, and/or a cytotoxic agent. Any appropriate method can be used to conjugate (e.g., covalently or non-covalently attach) another polypeptide or other moiety to a binder (e.g., an antibody, antigen binding fragment, and/or antibody domain) provided herein. For example, another polypeptide or other moiety can be conjugated to a binder (e.g., an antibody, antigen binding fragment, and/or antibody domain) provided herein using the methods described in U.S. Pat. No. 8,021,661.

In some cases, a binder (e.g., an antibody, antigen binding fragment, and/or antibody domain) provided herein can be modified with a moiety that improves its stabilization and/or retention in circulation, for example, in blood, serum, or other tissues by, for example, at least 1.5-, 2-, 5-, 10-, or 50-fold. For example, a binder (e.g., an antibody, antigen binding fragment, and/or antibody domain) provided herein can be attached (e.g., covalently or non-covalently attached) to a polymer such as a substantially non-antigenic polymer. Examples of substantially non-antigenic polymers that can be used as described herein include, without limitation, polyalkylene oxides and polyethylene oxides. In some cases, a polymer used herein can have any appropriate molecule weight. For example, a polymer having an average molecular weight from about 200 Daltons to about 35,000 Daltons (e.g., from about 1,000 to about 15,000 Daltons or from about 2,000 to about 12,500 Daltons) can be used. In some cases, a binder (e.g., an antibody, antigen binding fragment, and/or antibody domain) provided herein can be attached (e.g., covalently or non-covalently) to a water soluble polymer. Examples of water soluble polymers that can be used as described herein include, without limitation, hydrophilic polyvinyl polymers, polyvinylalcohol, polyvinylpyrrolidone, polyalkylene oxide homopolymers, polyethylene glycol (PEG), polypropylene glycols, polyoxyethylenated polyols, and copolymers thereof and/or block copolymers thereof provided that the water solubility of the copolymer or block copolymers is maintained.

In some cases, a binder (e.g., an antibody, antigen binding fragment, and/or antibody domain) provided herein can be attached (e.g., covalently or non-covalently attached) to one or more polyoxyalkylenes (e.g., polyoxyethylene, polyoxypropylene, or block copolymers of polyoxyethylene and polyoxypropylene), polymethacrylates, carbomers, branched or unbranched polysaccharides, or combinations thereof. For example, a binder (e.g., an antibody, antigen binding fragment, and/or antibody domain) provided herein can be covalently attached to polyoxyethylene.

This document also provides nucleic acid molecules (e.g., isolated nucleic acid molecules) having a nucleic acid sequence encoding at least part of a binder (e.g., an antibody, antigen binding fragment, and/or antibody domain) provided herein. For example, an isolated nucleic acid molecule provided herein can include a nucleic acid sequence encoding a heavy chain variable domain such as a heavy chain variable domain as set forth in FIG. 2A. In another example, an isolated nucleic acid molecule provided herein can include a nucleic acid sequence encoding a light chain variable domain such as a light chain variable domain as set forth in FIG. 2B. In another example, an isolated nucleic acid molecule provided herein can include a nucleic acid sequence encoding a VH domain such as a VH domain as set forth in FIG. 3. In some cases, an isolated nucleic acid molecule provided herein can include a nucleic acid sequence encoding both (a) a heavy chain variable domain and (b) a light chain variable domain, with or without, encoding a linker polypeptide set forth in FIG. 10. A nucleic acid provided herein (e.g., an isolated nucleic acid molecule) can be single stranded or double stranded nucleic acid of any appropriate type (e.g., DNA, RNA, or DNA/RNA hybrids).

This document also provides vectors (e.g., plasmid vectors or viral vectors) containing one or more nucleic acids provided herein. An example of a plasmid vector that can be designed to include one or more nucleic acids having a nucleic acid sequence encoding at least part of a binder (e.g., an antibody, antigen binding fragment, and/or antibody domain) provided herein includes, without limitation, phagemids. Examples of viral vectors that can be designed to include one or more nucleic acids having a nucleic acid sequence encoding at least part of a binder (e.g., an antibody, antigen binding fragment, and/or antibody domain) provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18 (2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding at least part of a binder (e.g., an antibody, antigen binding fragment, and/or antibody domain) provided herein.

In some cases, a vector (e.g., a plasmid vector or a viral vector) provided herein can include a nucleic acid sequence encoding scFv or antibody domain (e.g., a VH domain) provided herein.

A vector provided herein (e.g., a plasmid vector or viral vector provided herein) can include any appropriate promoter and other regulatory sequence (e.g., transcription and translation initiation and termination codons) operably linked the nucleic acid sequence encoding at least part of a binder (e.g., an antibody, antigen binding fragment, and/or antibody domain) provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, CMV promotors (e.g., immediate early CMV promotors), and AAV promoters.

Any appropriate method can be used to make a nucleic acid molecule (or vector such as a plasmid vector or viral vector) having a nucleic acid sequence encoding at least part of a binder (e.g., an antibody, antigen binding fragment, and/or antibody domain) provided herein. For example, molecule cloning techniques can be used to make a nucleic acid molecule (or vector such as a plasmid vector or viral vector) having a nucleic acid sequence encoding at least part of a binder (e.g., an antibody, antigen binding fragment, and/or antibody domain) provided herein as described elsewhere (see, e.g., Sambrook et al., Molecular Cloning: A Laboratory Manual, 2nd edition, Cold Spring Harbor Laboratory, NY (1989); and Ausubel et al., Current Protocols in Molecular Biology, Green Publishing Associates and John Wiley & Sons, New York, N.Y. (1994)).

This document also provides host cells that include a nucleic acid provided herein (e.g., a nucleic acid having a nucleic acid sequence encoding at least part of a binder (e.g., an antibody, antigen binding fragment, and/or antibody domain) provided herein). Host cells that can be designed to include one or more nucleic acids provided herein can be prokaryotic cells or eukaryotic cells. Examples of prokayotic cells that can be designed to include a nucleic acid provided herein include, without limitation, E. coli (e.g., Tb-1, TG-1, DH5α, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukayotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, chinese hamster ovary (CHO) cells, W138 BHK cells, COS-7 cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding at least part of a binder provided herein) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, NY (1989); Davis et al., Basic Methods in Molecular Biology (1986); and Neumann et al., EMBO J., 1:841 (1982)).

In some cases, a binder (e.g., an antibody, antigen binding fragment, and/or antibody domain) provided herein can be produced using a method that includes (a) introducing nucleic acid encoding the polypeptide into a host cell; (b) culturing the host cell in culture medium under conditions sufficient to express the polypeptide; (c) harvesting the polypeptide from the cell or culture medium; and (d) purifying the polypeptide (e.g., to reach at least 50, 60, 70, 80, 90, 95, 97, 98, or 99 percent purity).

In some cases, a binder (e.g., an antibody, antigen binding fragment, and/or antibody domain) provided herein, a nucleic acid provided herein (e.g., nucleic acid encoding an antibody, antigen binding fragment, and/or antibody domain provided herein), a vector provided herein (e.g., a viral vector designed to express an antibody, antigen binding fragment, and/or antibody domain provided herein), and/or a host cell provided herein (e.g., a host cell designed to express an antibody, antigen binding fragment, and/or antibody domain provided herein) can be formulated as a pharmaceutical composition for administration to a mammal (e.g., a human) having cancer to treat that mammal. In some cases, a binder (e.g., an antibody, antigen binding fragment, and/or antibody domain) provided herein, a nucleic acid provided herein (e.g., nucleic acid encoding an antibody, antigen binding fragment, and/or antibody domain provided herein), a vector provided herein (e.g., a viral vector designed to express an antibody, antigen binding fragment, and/or antibody domain provided herein), and/or a host cell provided herein (e.g., a host cell designed to express an antibody, antigen binding fragment, and/or antibody domain provided herein) can be formulated as a pharmaceutical composition for administration to a mammal (e.g. a human) to reduce the number of cancer cells within the mammal and/or to increase the survival of the mammal suffering from cancer. For example, a binder (e.g., an antibody, antigen binding fragment, and/or antibody domain) provided herein having the ability to bind to an NE polypeptide (e.g., a human NE polypeptide) can be formulated as a pharmaceutical composition for administration to a mammal (e.g. a human). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi, et al., Eur. J. Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include a binder (e.g., an antibody, antigen binding fragment, and/or antibody domain) provided herein (or a nucleic acid, a vector, or a host cell provided herein) can be formulated to include a buffer (e.g., an acetate, citrate, histidine, succinate, phosphate, or hydroxymethylaminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), anticancer agents such as enzalutamide, imanitib, gefitinib, erlotini, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, obatoclax, navitoclax, taxol, paclitaxel, or bevacizumab, or combinations thereof. For example, a pharmaceutical composition provided herein can be formulated to include one or more binders (e.g., one or more antibodies, one or more antigen binding fragments, and/or one or more antibody domains) provided herein in combination with one or more checkpoint inhibitors such as anti-PD-1 antibodies or PD-1 inhibitors (e.g., cemiplimab, nivolumab, pembrolizumab, JTX-4014, spartalizumab, camrelizumab, sintilimab, tislelizumab, toripalimab, dostarlimab, INCMGA00012, AMP-224, or AMP-514), anti-PD-L1 antibodies or PD-L1 inhibitors (e.g., avelumab, durvalumab, atezolizumab, KN035, CK-301, AUNP12, CA-170, or BMS-986189), and/or anti-CTLA-4 antibodies (e.g., ipilimumab).

In some cases, when a pharmaceutical composition is formulated to include one or more binders (e.g., one or more antibodies, one or more antigen binding fragments, and/or one or more antibody domains) provided herein, any appropriate concentration of the binder can be used. For example, a pharmaceutical composition provided herein can be formulated to be a liquid that includes from about 1 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a binder (e.g., an antibody, antigen binding fragment, and/or antibody domain) provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a binder (e.g., an antibody, antigen binding fragment, and/or antibody domain) provided herein. In some cases, a pharmaceutical composition containing a binder (e.g., an antibody, antigen binding fragment, and/or antibody domain) provided herein can be formulated as a dosage form with a titer of the binder being from about 1×10⁵ to about 1×10¹² (e.g., from about 1×10⁵ to about 1×10¹⁰, from about 1×10⁵ to about 1×10⁸, from about 1×10⁶ to about 1×10¹², from about 1×10⁶ to about 1×10¹², from about 1×10⁸ to about 1×10¹², from about 1×10⁹ to about 1×10¹², from about 1×10⁶ to about 1×10¹¹, or from about 1×10⁷ to about 1×10¹⁰).

In some cases, when a pharmaceutical composition is formulated to include one or more nucleic acids (e.g., vectors such as viral vectors) encoding at least part of a binder (e.g., an antibody, antigen binding fragment, and/or antibody domain) provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid provided herein.

In some cases, a pharmaceutical composition designed to include a binder (e.g., an antibody, antigen binding fragment, and/or antibody domain) provided herein can be formulated to include one or more agents capable of reducing aggregation of the binder when formulated. Examples of such agents that can be used as described herein include, without limitation, methionine, arginine, lysine, aspartic acid, glycine, glutamic acid, and combinations thereof. In some cases, one or more of these amino acids can be included within the formulation at a concentration from about 0.5 mM to about 145 mM (e.g., from about 1 mM to about 145 mM, from about 10 mM to about 145 mM, from about 100 mM to about 145 mM, from about 0.5 mM to about 125 mM, from about 0.5 mM to about 100 mM, from about 0.5 mM to about 75 mM, or from about 10 mM to about 100 mM).

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more binders (e.g., one or more antibodies, one or more antigen binding fragments, and/or one or more antibody domains) provided herein can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides methods for administering a composition (e.g., a pharmaceutical composition provided herein) containing one or more binders (e.g., one or more antibodies, one or more antigen binding fragments, and/or one or more antibody domains) provided herein (or a nucleic acid, vector, or host cell provided herein) to a mammal (e.g., a human). For example, a composition (e.g., a pharmaceutical composition provided herein) containing one or more binders (e.g., one or more antibodies, one or more antigen binding fragments, and/or one or more antibody domains) provided herein (or a nucleic acid, vector, and/or host cell provided herein) can be administered to a mammal (e.g., a human) having cancer to treat that mammal. In some cases, a composition (e.g., a pharmaceutical composition provided herein) containing one or more binders (e.g., one or more antibodies, one or more antigen binding fragments, and/or one or more antibody domains) provided herein (or a nucleic acid, vector, and/or host cell provided herein) can be administered to a mammal (e.g. a human) to reduce the number of cancer cells within the mammal and/or to increase the survival of the mammal suffering from cancer.

Any appropriate cancer can be treated using a composition (e.g., a pharmaceutical composition provided herein) containing one or more binders (e.g., one or more antibodies, one or more antigen binding fragments, and/or one or more antibody domains) provided herein (or a nucleic acid, vector, or host cell provided herein). For example, a mammal (e.g., a human) having cancer can be treated by administering a composition (e.g., a pharmaceutical composition) containing one or more binders (e.g., one or more antibodies, one or more antigen binding fragments, and/or one or more antibody domains) provided herein to that mammal. Examples of cancers that can be treated as described herein include, without limitation, lung cancer, prostate cancer, esophageal cancer, stomach cancer, colorectal cancer, liver cancer, vaginal cancer, cervical cancer, pancreatic cancer, head and neck cancer, blood cancer, skin cancer, brain cancer, bone cancer, and breast cancer. In some cases, a mammal (e.g., a human) having a cancer (e.g., a lung cancer, a prostate cancer, an esophageal cancer, a stomach cancer, a colorectal cancer, a liver cancer, a vaginal cancer, or a cervical cancer) can be administered a composition (e.g., a pharmaceutical composition) containing one or more binders (e.g., one or more antibodies, one or more antigen binding fragments, and/or one or more antibody domains) provided herein to treat that mammal (e.g., to reduce the number of cancer cells within the mammal).

Any appropriate inflammatory condition can be treated using a composition (e.g., a pharmaceutical composition provided herein) containing one or more binders (e.g., one or more antibodies, one or more antigen binding fragments, and/or one or more antibody domains) provided herein (or a nucleic acid, vector, or host cell provided herein). For example, a mammal (e.g., a human) having COPD, cystic fibrosis, acute lung injury, or acute respiratory distress syndrome can be treated by administering a composition (e.g., a pharmaceutical composition) containing one or more binders (e.g., one or more antibodies, one or more antigen binding fragments, and/or one or more antibody domains) provided herein to that mammal.

Any appropriate method can be used to administer a composition (e.g., a pharmaceutical composition) provided herein to a mammal (e.g., a human). For example, a composition provided herein (e.g., a pharmaceutical composition containing one or more binders provided herein such as one or more antibodies, one or more antigen binding fragments, and/or one or more antibody domains provided herein) can be administered to a mammal (e.g., a human) intravenously (e.g., via an intravenous injection or infusion), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) can be adjusted for the mammal being treated.

In some cases, an effective amount of a composition containing one or more binders (e.g., one or more antibodies, one or more antigen binding fragments, and/or one or more antibody domains) provided herein (or a nucleic acid, vector, or host cell provided herein) (e.g., a pharmaceutical composition provided herein) can be an amount that reduces the number of cancer cells and/or reduces inflammation within a mammal having cancer and/or an inflammatory condition without producing significant toxicity to the mammal. In some cases, an effective amount of a composition containing one or more binders (e.g., one or more antibodies, one or more antigen binding fragments, and/or one or more antibody domains) provided herein (or a nucleic acid, vector, or host cell provided herein) (e.g., a pharmaceutical composition provided herein) can be an amount that increases the survival time of a mammal having cancer (and/or an inflammatory condition) as compared to a control mammal having comparable cancer (and/or an inflammatory condition) and not treated with the composition. For example, an effective amount of a binder (e.g., an antibody, antigen binding fragment, and/or antibody domain) provided herein can be from about 0.001 mg/kg to about 100 mg/kg (e.g., from about 0.001 mg/kg to about 90 mg/kg, from about 0.001 mg/kg to about 80 mg/kg, from about 0.001 mg/kg to about 70 mg/kg, from about 0.001 mg/kg to about 60 mg/kg, from about 0.001 mg/kg to about 50 mg/kg, from about 0.001 mg/kg to about 40 mg/kg, from about 0.001 mg/kg to about 30 mg/kg, from about 0.005 mg/kg to about 100 mg/kg, from about 0.01 mg/kg to about 100 mg/kg, from about 0.05 mg/kg to about 100 mg/kg, from about 0.1 mg/kg to about 100 mg/kg, from about 0.5 mg/kg to about 100 mg/kg, from about 1 mg/kg to about 100 mg/kg, from about 5 mg/kg to about 100 mg/kg, from about 0.01 mg/kg to about 25 mg/kg, from about 0.1 mg/kg to about 30 mg/kg, from about 0.15 mg/kg to about 25 mg/kg, from about 0.2 mg/kg to about 20 mg/kg, from about 0.5 mg/kg to about 20 mg/kg, from about 1 mg/kg to about 30 mg/kg, from about 1 mg/kg to about 25 mg/kg, from about 1 mg/kg to about 20 mg/kg, from about 2 mg/kg to about 20 mg/kg, from about 5 mg/kg to about 30 mg/kg, from about 10 mg/kg to about 30 mg/kg, from about 15 mg/kg to about 30 mg/kg, from about 20 mg/kg to about 30 mg/kg, from about 3 mg/kg to about 30 mg/kg, from about 0.5 mg/kg to about 10 mg/kg, from about 1 mg/kg to about 10 mg/kg, from about 1 mg/kg to about 5 mg/kg, or from about 1 mg/kg to about 3 mg/kg). The effective amount can remain constant or can be adjusted as a sliding scale or variable dose depending on the mammal's response to treatment. Various factors can influence the actual effective amount used for a particular application. For example, the severity of cancer when treating a mammal having cancer, the route of administration, the severity of an inflammatory condition when treating a mammal having an inflammatory condition, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective amount of a composition provided herein (e.g., a pharmaceutical composition containing one or more binders provided herein) that is administered.

In some cases, an effective frequency of administration of a composition containing one or more binders (e.g., one or more antibodies, one or more antigen binding fragments, and/or one or more antibody domains) provided herein (or a nucleic acid, vector, or host cell provided herein) (e.g., a pharmaceutical composition provided herein) can be a frequency that reduces the number of cancer cells (and/or inflammation) within a mammal having cancer (and/or an inflammatory condition) without producing significant toxicity to the mammal. In some cases, an effective frequency of administration of a composition containing one or more binders (e.g., one or more antibodies, one or more antigen binding fragments, and/or one or more antibody domains) provided herein (or a nucleic acid, vector, or host cell provided herein) (e.g., a pharmaceutical composition provided herein) can be a frequency that increases the survival time of a mammal having cancer (and/or an inflammatory condition) as compared to a control mammal having comparable cancer (and/or an inflammatory condition) and not treated with the composition. For example, an effective frequency of administration of a pharmaceutical composition provided herein such as a pharmaceutical composition containing one or more binders provided herein can be from about twice daily to about once a year (e.g., from about twice daily to about once a month, from about twice daily to about once a week, from about once daily to about once a month, or from one once daily to about once a week). In some cases, the frequency of administration of a pharmaceutical composition provided herein such as a pharmaceutical composition containing one or more binders provided herein can be daily. The frequency of administration of a pharmaceutical composition provided herein such as a pharmaceutical composition containing one or more binders provided herein can remain constant or can be variable during the duration of treatment. Various factors can influence the actual effective frequency used for a particular application. For example, the severity of a cancer, the severity of an inflammatory condition, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective frequency of administration of a composition provided herein (e.g., a pharmaceutical composition containing one or more binders provided herein).

In some cases, an effective duration of administration of a composition containing one or more binders (e.g., one or more antibodies, one or more antigen binding fragments, and/or one or more antibody domains) provided herein (or a nucleic acid, vector, or host cell provided herein) (e.g., a pharmaceutical composition provided herein) can be a duration that reduces the number of cancer cells (and/or inflammation) within a mammal without producing significant toxicity to the mammal. In some cases, an effective duration of administration of a composition containing one or more binders (e.g., one or more antibodies, one or more antigen binding fragments, and/or one or more antibody domains) provided herein (or a nucleic acid, vector, or host cell provided herein) (e.g., a pharmaceutical composition provided herein) can be a duration that increases the survival time of a mammal having cancer (and/or an inflammatory condition) as compared to a control mammal having comparable cancer (and/or an inflammatory condition) and not treated with the composition. For example, an effective duration of administration of a pharmaceutical composition provided herein such as a pharmaceutical composition containing one or more binders provided herein can vary from a single time point of administration to several weeks to several months (e.g., 4 to 12 weeks). Multiple factors can influence the actual effective duration used for a particular application. For example, the severity of a cancer, the severity of an inflammatory condition, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective duration of administration of a composition provided herein (e.g., a pharmaceutical composition containing one or more binders provided herein).

In some cases, a binder (e.g., an antibody, antigen binding fragment, and/or antibody domain) provided herein can be used to detect the presence or absence of an NE polypeptide (e.g., a human NE polypeptide) in vitro, in situ, or in vivo (e.g., in vivo imaging within a mammal such as a human). For example, a binder (e.g., an antibody, antigen binding fragment, and/or antibody domain) provided herein can be designed to include a label (e.g., a covalently attached radioactive, enzymatic, colorimetric, or fluorescent label). The labelled binder can be used to detect the presence or absence of an NE polypeptide (e.g., a human NE polypeptide) within a biological sample in vitro. Examples of biological samples that can be assessed using a binder (e.g., an antibody, antigen binding fragment, and/or antibody domain) provided herein include, without limitation, serum samples, plasma samples, tissue samples, biopsy samples, cell line samples, and tissue culture samples. In some cases, a biological sample that can be assessed as described herein can include mammalian body tissues and/or cells such as leukocytes, T cells, NK cells, ovary tissue or cells, prostate tissue or cells, heart tissue or cells, placenta tissue or cells, pancreas tissue or cells, liver tissue or cells, spleen tissue or cells, lung tissue or cells, breast tissue or cells, head and neck tissue or cells, endometrium tissue or cells, colon tissue or cells, colorectal tissue or cells, cervix tissue or cells, stomach tissue or cells, or umbilical tissue or cells that may express an NE polypeptide (e.g., a human NE polypeptide). In some cases, a binder (e.g., an antibody, antigen binding fragment, and/or antibody domain) provided herein can be immobilized, e.g., on a support, and retention of an NE polypeptide (e.g., a human NE polypeptide) from a biological sample on the support can be detected, and/or vice versa. In some cases, a binder (e.g., an antibody, antigen binding fragment, and/or antibody domain) provided herein can be used in applications such as fluorescence polarization, microscopy, ELISA, centrifugation, chromatography, and/or cell sorting (e.g., fluorescence activated cell sorting).

In some cases, a binder (e.g., an antibody, antigen binding fragment, and/or antibody domain) provided herein containing a label (e.g., a covalently attached radioactive label) can be used to detect the presence or absence of an NE polypeptide (e.g., a human NE polypeptide) within a mammal (e.g., a human). For example, a binder (e.g., an antibody, antigen binding fragment, and/or antibody domain) provided herein that is labelled (e.g., covalently labelled) with a radiolabel or an MRI detectable label can be administered to a mammal (e.g., a human), and that mammal can be assessed using a means for detecting the detectable label. In some cases, a mammal can be scanned to evaluate the location(s) of a labelled binder provided herein within the mammal. For example, the mammal can be imaged using NMR or other tomographic techniques.

Examples of labels that can be attached (e.g., covalently or non-covalently attached) to a binder (e.g., an antibody, antigen binding fragment, and/or antibody domain) provided herein include, without limitation, radiolabels such as ¹³¹I, ¹¹¹In, ¹²³I, ^{99m}Tc, ³²p, ³³p, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as a peroxidase or a phosphatase. In some cases, short-range radiation emitters such as isotopes detectable by short-range detector probes can be used.

The invention will be further described in the following examples, which do not limit the scope of the invention described in the claims.

### EXAMPLES

### Example 1—Obtaining Binders Having the Ability to Bind to a Human NE Polypeptide

A vector was generated to express an NE polypeptide having amino acid residues S28 to H267 (SEQ ID NO: 166) of a human NE polypeptide fused to human IgG₁ Fc (see, e.g., FIG. 1). This NE-IgG₁ fusion polypeptide was used to identify an Fab antibody fragment (Clone #1; also referred to as 1C10; FIGS. 2A-B) and an VH domain (Clone #2; also referred to as 1D1.43; FIG. 3).

### Materials and Methods

Protein Expression and Purification (E. coli and Expi 293 Cell)

ELANE gene was synthesized by IDT (Coralville, Iowa) and cloned into pSecTag expression vector, which fused NE polypeptide with human IgG₁ Fc. For the conversion of IgG₁ from Fab, the heavy chain and light chain of Fab were amplified and re-cloned into the pcDNA-IgG₁ vector. For transient expression, the plasmid was transfected into Ex-pi293 cells by PEI, and purified by Protein A resin (GenScript). The expression and purification of VH and Fab binders were performed in E. coli Top10F′ bacteria with 1 mM IPTG induction at 30° C. for 16 hours. Bacterial pellets were lysed by Polymyxin B (Sigma-Aldrich), and the supernatant was loaded on Ni-NTA column (GE Healthcare) for purification.

Panning and Screening from Two Large Phage Library

To select antibodies against neutrophil elastase, two large VH and Fab phage libraries were used for panning separately. Briefly, 10 μg NE-Fc were coated in 96-well Protein G microplate in PBS at 4° C. overnight. The Protein G plate was blocked with 5% milk for 1 hour at room temperature, and then the phage library was added into the NE-Fc coated Protein G plate. The plates were incubated at room temperature for 1 hour, and then washed with PBST. After washing, phages were eluted by 0.1M, PH3 Glycine, and neutralized with 1 M, pH8 Tris-HCl. For the second and third round panning, 5 μg and 2.5 μg of NE-Fc were used as antigen. After the third round panning, 192 individual clones were screened for binding to NE-His protein by soluble phage ELISA, and 11 unique Fab and 4 VH were identified.

### Size-Exclusion Chromatography (SEC)

The purity and structure of the antibodies were analyzed by Superdex 200 Increase 10/300 GL chromatography (GE Healthcare). The standard proteins used for calibration and their molecular weights were: Ferritin (Mᵣ 440 kDa), Aldolase (Mᵣ 158 kDa), Conalbumin (Mᵣ 75 kDa), Ovalbumin (Mᵣ 44 kDa), Carbonic anhydrase (Mᵣ 29 kDa), and Ribonuclease A (Mᵣ 13.7 kDa) at 3 mg/mL. A 500 μL sample mix containing the above proteins were loaded to the column and separated by the ÄKTA explorer machine (GE Healthcare). For the antibody analysis, 100 μL of filtered antibodies (2 mg/mL) in 1×DPBS (Dulbecco's phosphate-buffered saline, Gibco) were analyzed. Antibody was eluted by DPBS buffer at a flow rate of 0.5 mL/minute.

### Dynamic Light Scattering (DLS)

The aggregation resistance of VH 1D1.43 and Fab 1C10 was measured by dynamic light scattering (DLS). The buffer was changed to DPBS and filtered by a 0.22 μm filter. The antibody concentration was adjusted to 1 mg/mL. 500 μL antibody sample was incubated at 37° C. without shaking. On day 0 and day 7, samples were taken for DLS measurements on Zetasizer Nano ZS ZEN3600 (Malvern Instruments Limited, Westborough, MA) to determine the antibody size distribution.

### Enzyme-Linked Immunosorbent Assay (ELISA)

The NE polypeptide was coated in 96-well microplates at 50 ng/well in PBS at 4° C. over-night and blocked with 5% milk in PBS for 2 hours at room temperature. For the soluble Fab/VH binding assay, the 3-fold serially diluted Fab/VH were added and incubated for 1 hour at 37° C. and further incubated with anti-FLAG M2-peroxidase (HRP) antibody (A8592, Sigma-Aldrich) for another 1 hour. For the IgG₁ binding assay, HRP conjugated goat anti-human IgG₁ Fc (Sigma-Aldrich) was used for detection. Finally, the reaction was developed by 3,3′,5,5′-tetramethylbenzidine (TMB, Sigma-Aldrich) and stopped by TMB stop buffer (ScyTek laboratories). Absorbance was recorded at 450 nm. The experiment was performed in duplicate.

### BLItz

The affinity and avidity of anti-hNE binders were detected by biolayer interferometry BLItz (ForteBio, Menlo Park, CA). Briefly, Dulbecco's PBS (DPBS) was used to establish baseline for 30 seconds, and streptavidin biosensor (ForteBio) was coated with 16.7 μg/mL recombinant NE-Biotin for 2 minutes. Different concentrations of Fab and IgG₁ were used for association, which was monitored for 2 minutes to measure the affinity and avidity. Antigen coated biosensors with PBS served as reference controls. The dissociation was monitored in DPBS for 4 minutes.

### Enzyme Activity Inhibition Assay

The inhibition effects of antibodies on enzyme activity were determined by an Elastase Inhibitor Screening Kit (Sigma-Aldrich). Briefly, binders were diluted to the desired concentration by assay buffer, 50 μL of neutrophil elastase solution was added, and the reaction incubated at 37° C. for 45 minutes. The SPCK NE inhibitor was used as a positive control. After incubation, 25 μL substrate solution were add to each reaction well, and the fluorescence was measure in kinetic mode for 30 minutes. The result was calculated by % Relative inhibition.

### Cells and Flow Cytometry

The human prostate adenocarcinoma cell line PC-3 was purchased from ATCC. The cells were maintained in F-12k medium plus 10% FBS and 1% penicillin/streptomycin. Cells were seeded at a concentration of 1×10⁵ cells/well in a 24-well plate and allowed to adhere overnight and then incubated in serum-free medium for 24 hours. Neutrophil elastase was labeled with FITC by a FITC protein labeling kit (Thermo Fisher) according to the manufacturer's instructions. Cells were treated with 30 nM NE-FITC with or without 100 nM binders for 1 hour, and the cells were analyzed by flow cytometry (BD Bioscience). The data were analyzed using FlowJo Software.

### Wound Healing Assay

LL 47 fibroblast cells were seeded into 24-well plate and grown to confluence in complete F-12K medium. After being starved for 1 hour in serum-free medium, the cells were wounded with 200 μL pipet tips, washed with PBS, and treated with NE with or without binders at different doses for 24 hours. Images were captured under a 4× objective microscope (Olympus microscope). Wound areas were quantified by using Image J software.

### Conformational Epitope Mappings

Conformational epitope mapping was proceeded by PEPperPRINT GanbH (Heidelberg, Germany). Briefly, the sequence of hNE was elongated with neutral GSGSGSG linkers at the C- and N-terminus. The elongated antigen sequence was converted into 7, 10, and 13 amino acid peptides with peptide-peptide overlaps of 6, 9, and 12 amino acids. Human antibodies IgG1 1C10 and VH-Fc 1D1.43 at a concentration of 10 μg/mL and 30 μg/mL were incubated with antigen peptides for 16 hours at 4° C. followed by goat anti-human IgG DyLight680 incubation for 45 minutes at room temperature. The result was read by Innopsys InnoScan 710-IR Microarray Scanner.

### Molecular Docking for Predicting Binding Models of Fab 1C10 and VH 1D1.43

Fab 1C10 and VH 1D1.43 structures were modeled in Swiss-Model followed by energy minimization. Z-DOCK program was used for docking Fab 1C10 and VH 1D1.43 onto NE, which structure was resolved as (PDB: 3Q76). The antibody binding and blocking regions on NE were based on the experimental epitope mapping results. Z-DOCK output top 10 optimal poses, which were visually scrutinized for interaction interface compatibility and side chain clashes, with selections of the most favorable pose as the binding models. The structural figures were prepared by PyMol 2.5.

### Statistical Analysis

Statistical analyses including EC₅₀ and IC₅₀ were performed by GraphPad Prism. Experiments were repeated a minimum of three times. Differences were considered statistically significant when p<0.05.

### Results

### Selection of High-Affinity VH Domains and Fab Antibodies Against Neutrophil Elastase

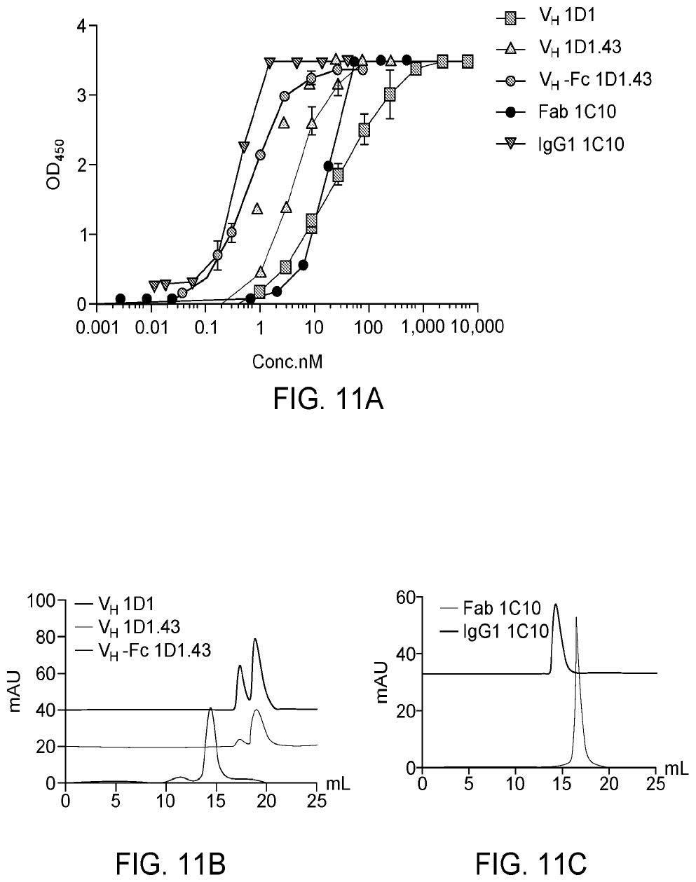

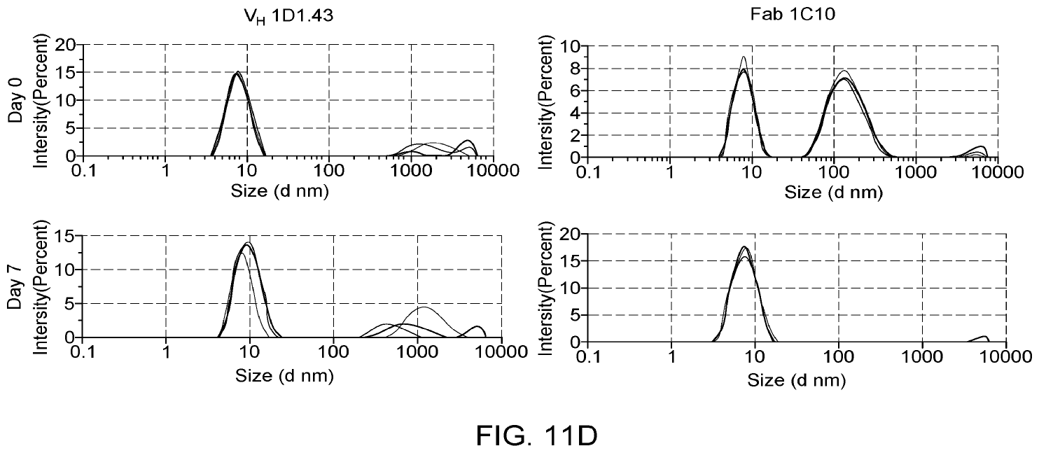

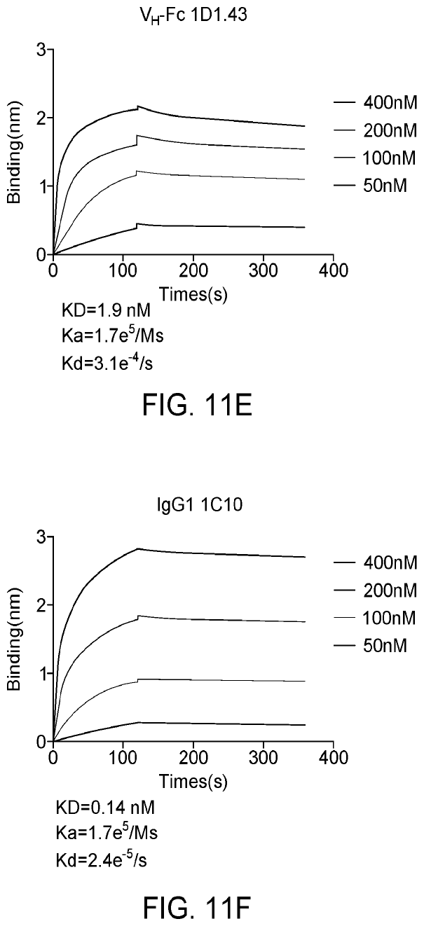

Several large phage-displayed libraries were developed using PBMC from non-immunized healthy donors (Sun et al., MAbs, 12 (1): 1778435 (2020); and Li et al., Cell, 183 (2): 429-441e16 (2020)). In this study, two large phage-displayed human VH and Fab libraries were used for panning against recombinant NE protein, which was fuse with the human IgG1 Fc tag due to the enhanced stability. After three rounds of panning, a panel of VH and Fab binders were screened. Among these binders, two high-affinity antibodies, termed as Fab 1C10 and VH 1D1, were selected based on their high-affinities and favorable biophysical properties. The EC₅₀ values of Fab 1C10 and VH 1D1 tested by ELISA were 16.3±0.52 nM and 25.5±2.57 nM, respectively (FIG. 11A). The equilibrium dissociation constant (KD) of these two binders (Fab 1C10 and VH 1D1) with recombinant human ELA2 measured by BLItz were 8.5 nM and 69.7 nM, respectively (Table A). To further improve the affinity of the VH 1D1 binder, a random mutant library was constructed by using error-prone PCR. After three rounds of panning against NE-Fc with kinetic selection by using the prototype VH 1D1, one clone named as VH 1D1.43 was identified. The EC₅₀ value of VH 1D1.43 tested by ELISA was 4.3±0.17 nM (FIG. 11A), and the equilibrium dissociation constant was 8.4 nM, 5-10 folds higher than that of the parental clone (Table A). In addition, the aggregation propensity of these binders was evaluated by using dynamic light scattering (DLS) and size exclusion chromatography (SEC). The VH 1D1 has both monomer and dimer forms, but after affinity maturation, the main form of VH 1D1.43 was a monomer measured by SEC (FIG. 11B), and there was no obvious aggregation after 7 days incubation at 37° C. as measured by DLS (FIG. 11D). Fab 1C10 was a monomer and had no high molecular weight forms measured by SEC (FIG. 11C). Even though Fab 1C10 showed a high molecular peak by DLS at 4° C., but after 7 days incubation at 37° C. at a concentration of 1 mg/mL, there was no aggregation observed by DLS, indicating that the high molecular form was reversible and soluble (FIG. 11D).

TABLE A
Blitz results of human neutrophil elastase antibodies.

| | kon | koff | K_{D} |
|---|---|---|---|
| Antibody | (M⁻¹Sec⁻¹) | (Sec⁻ ¹) | (nM) |
| V_{H} 1D1 | 5.9 × 10⁴ | 4.1 × 10⁻³ | 69.7 |
| V_{H} 1D1.43 | 7.2 × 10⁴ | 6 × 10⁻⁴ | 8.4 |
| V_{H}-Fc 1D1.43 | 1.7 × 10⁵ | 3.1 × 10⁻⁴ | 1.9 |
| Fab 1C10 | 4.9 × 10⁴ | 4.1 × 10⁻⁴ | 8.5 |
| IgG1 1C10 | 1.7 × 10⁵ | 2.4 × 10⁻⁵ | 0.14 |

### Conversion of VH Domain and Fab Binders to VH-Fc and IgG1

To increase the avidity and extend the in vivo half-life of VH 1D1.43 and Fab 1C10, these two binders were converted to a bivalent antibody by fusion to the human IgG1 Fc (VH-Fc 1D1.43 and IgG1 1C10). The EC₅₀ values of IgG1 1C10 and VH-Fc 1D1.43 tested by ELISA were 0.4±0.02 nM and 0.66±0.03 nM, respectively (FIG. 11A). The equilibrium dissociation constant (KD) of VH-Fc 1D1.43 and IgG1 1C10 measured by BLItz were 1.9 nM and 0.14 nM, respectively (FIGS. 11E and 11F; Table A). The forms of IgG 1C10 and VH-Fc 1D1.43 were monomers and had no high molecular weight species measured by SEC (FIGS. 11B and 11C).

Specificity of VH Domain and Fab Binders with Human NE

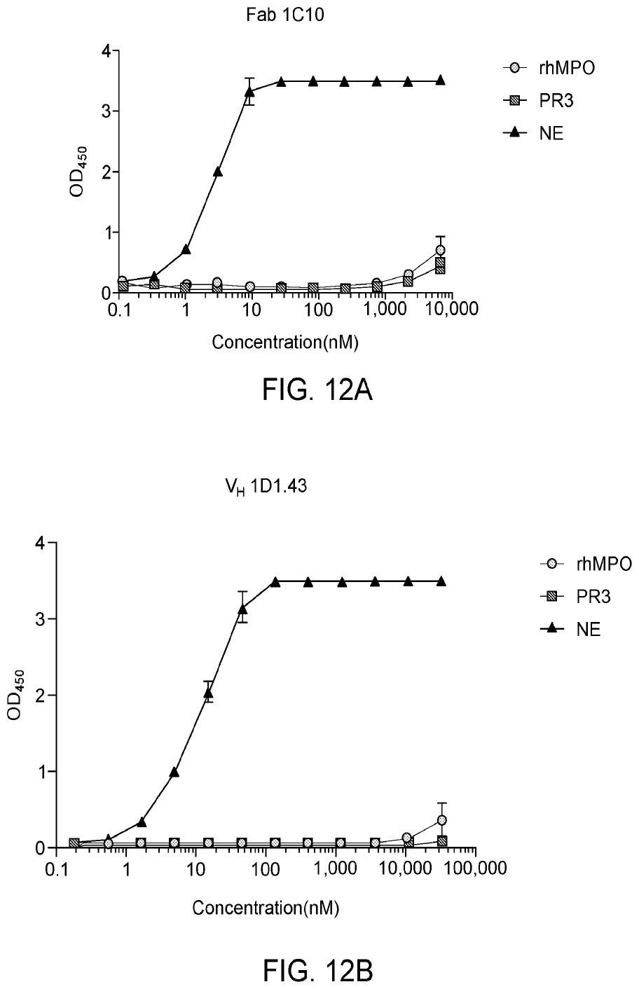

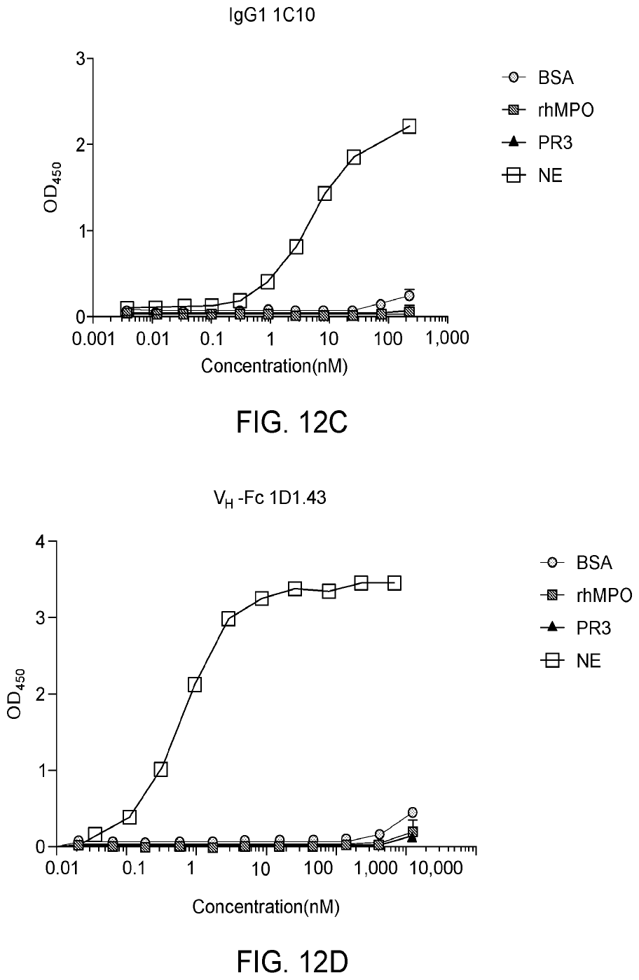

To further test the specificity of these two VH domain and Fab binders, the binding effects with recombinant human myeloperoxidase protein (rhMPO) and Proteinase 3 (PR3), which both were released by neutrophils, were tested. The ELISA results showed that Fab 1C10 and VH 1D1.43 specifically bound to NE and did not bind to other enzymes released by neutrophils (FIGS. 12A and 12B). The specificity of IgG1 1C10 and VH-Fc 1D1.43 was tested by detection of binding to BSA. The results showed that the binders had no non-specific binding with other proteins (FIGS. 12C and 12D).

### Epitope Mapping of IgG1 IC10 and VH-Fc ID1.43

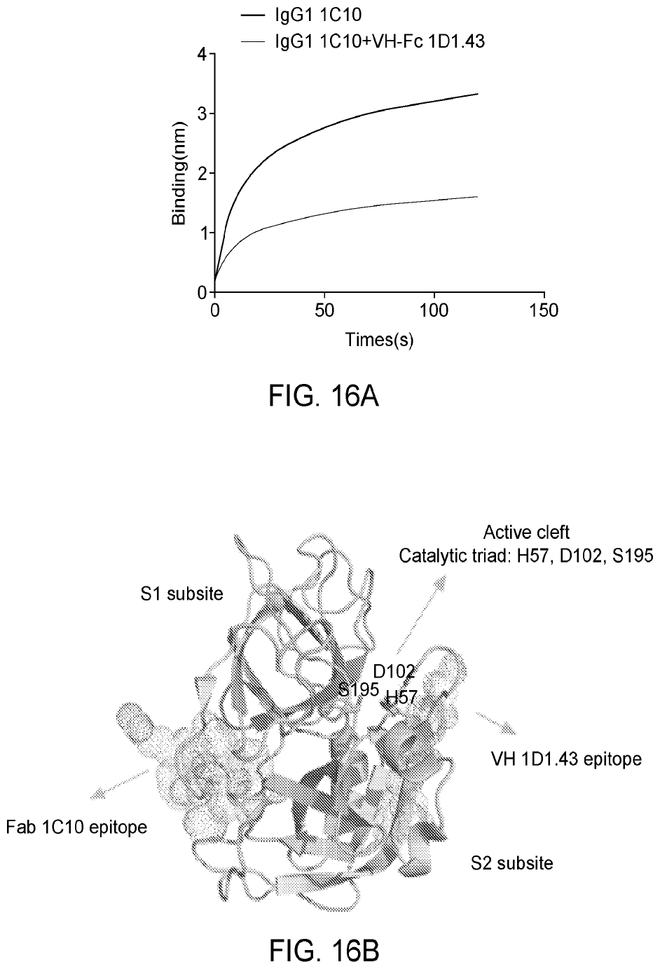

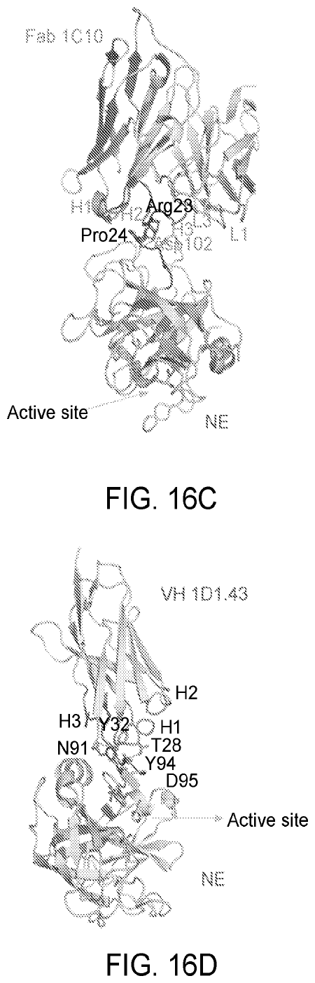

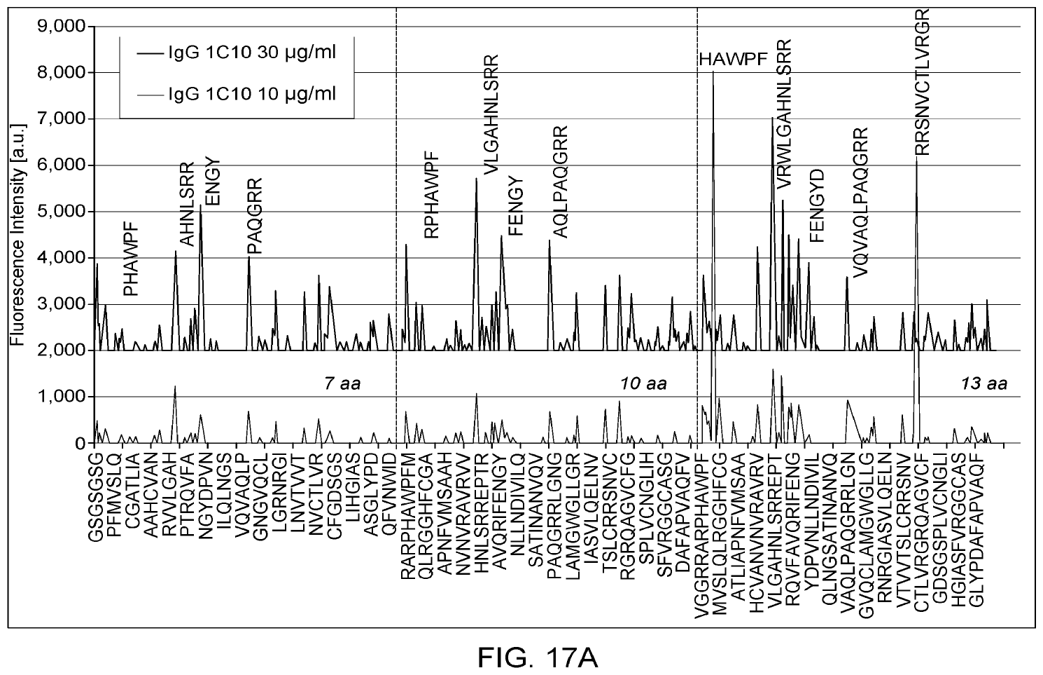

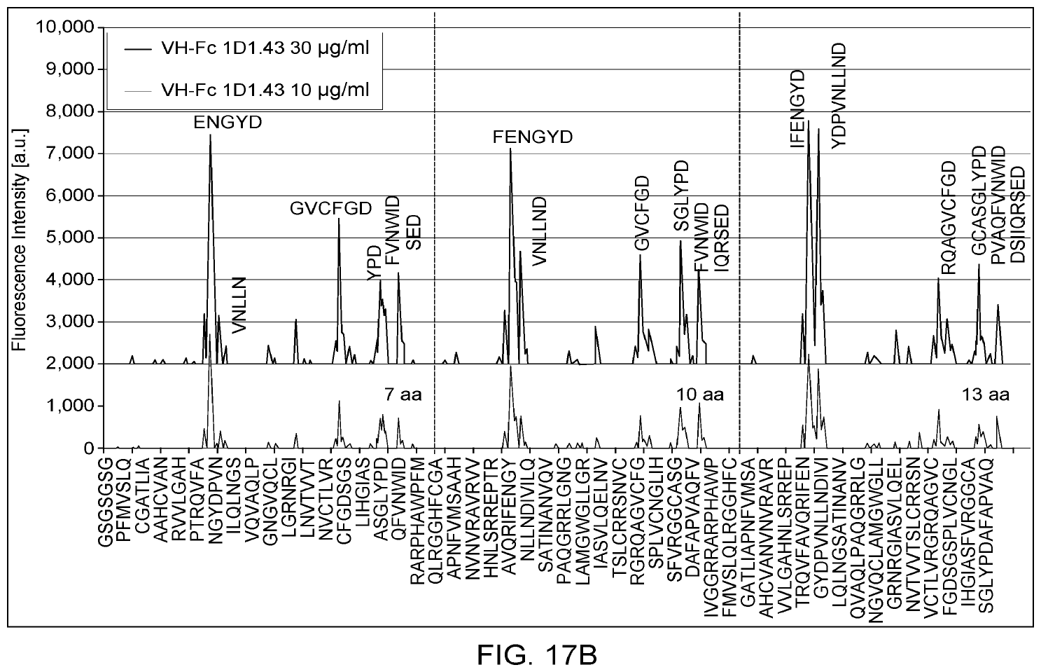

To test the binding epitope of IgG1 1C10 and VH-Fc 1D1.43, competition Blitz and peptide array based conformational epitope mapping was performed. The competition effects of IgG1 1C10 and VH-Fc 1D1.43 for binding to human NE were tested by BLItz (FIG. 16A). It showed that IgG1 1C10 and VH-Fc 1D1.43 did not compete with each other, suggesting that the epitope of these two binders are different. This was confirmed by conformational epitope mapping (FIG. 16B and FIG. 17). The conformational epitope mapping result showed that IgG1 1C10 had the antibody response against epitope-like spot patterns formed by adjacent lengths with the consensus motifs RPHAWPF (SEQ ID NO:167), VRVVLGAHNLSRR (SEQ ID NO:168), FENGYD (SEQ ID NO:169), and VQVAQLPAQGRR (SEQ ID NO:170), at all peptide lengths. But the interactions with peptides with the consensus motifs VRVVLGAHNLSRR (SEQ ID NO:171), VQVAQLPAQGRR (SEQ ID NO:172), and RRSNVCTLVRGR (SEQ ID NO:173) likely resulted from non-specific ionic antibody binding due to shorter basic RR or RGR motifs in these consensus motifs. And motif RPHAWPF (SEQ ID NO:174) showed clearer spot morphologies than motif FENGYD (SEQ ID NO:175) (FIG. 17A). Therefore, it was predicted that IgG1 1C10 binds to an epitope motif of ‘RPHAWPF’ (SEQ ID NO:176) which was located at the connecting hinge region of NE S1 and S2 subsites and was distal from the NE active site as defined by the catalytic triad (H57-D102-S195) (FIG. 16C), indicating that the inhibition effects of IgG1 1C10 on NE may be triggered by the NE conformational change upon its binding. By contrast, VH-Fc 1D1.43 showed high antibody response to the ‘IFENGYD’ (SEQ ID NO: 177) motif at all peptide lengths, which was adjacent to the active site (FIGS. 16D and 17B), indicating that the inhibition effect of VH-Fc 1D1.43 may be due to directly blocking accession of the substrate to the active site.

### Inhibition on NE Enzyme Activity and Function

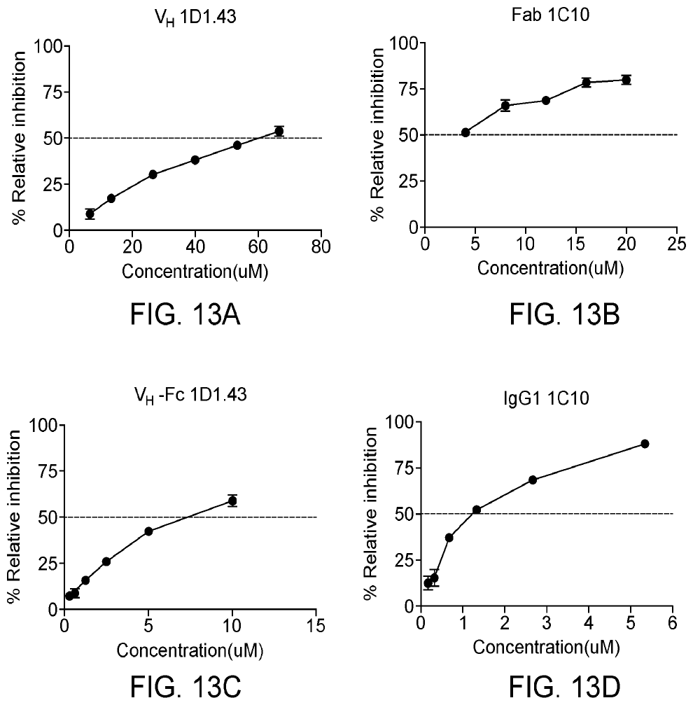

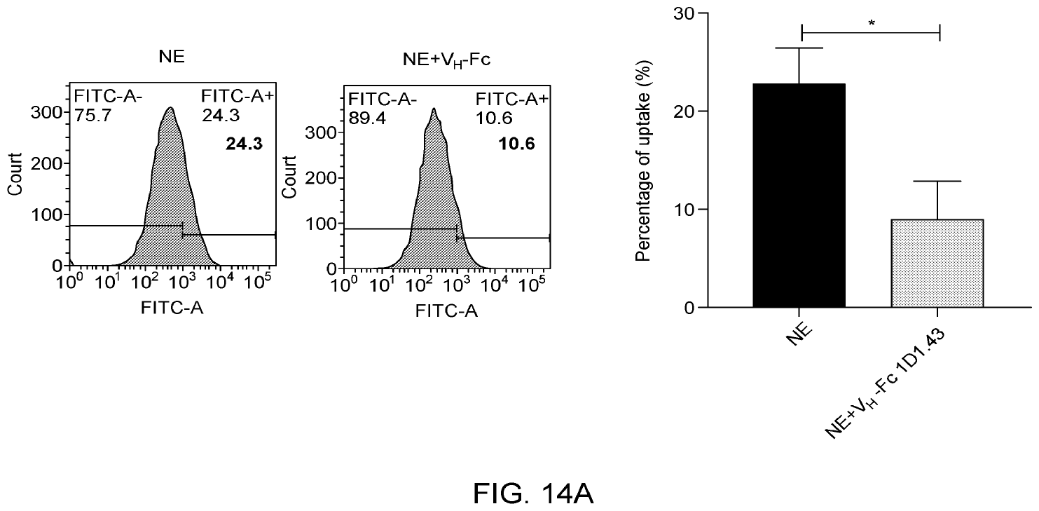

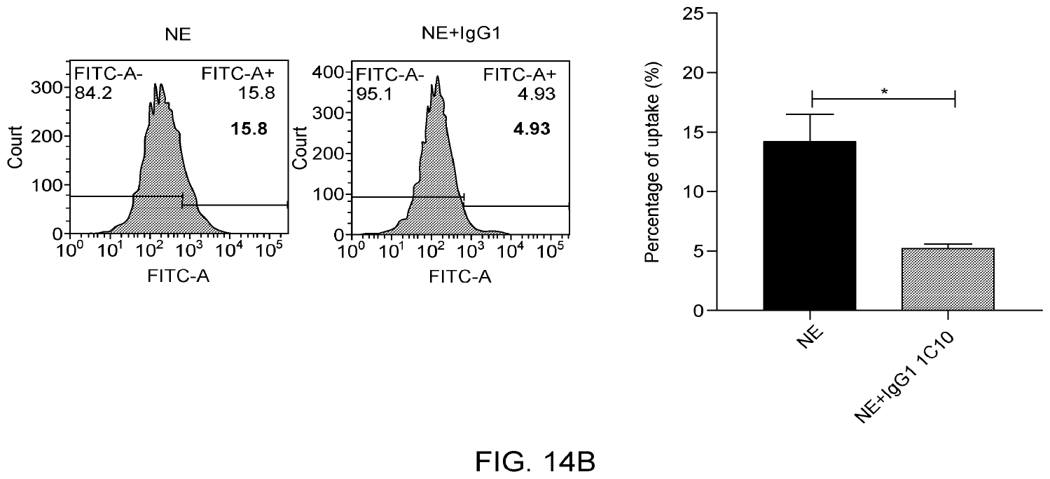

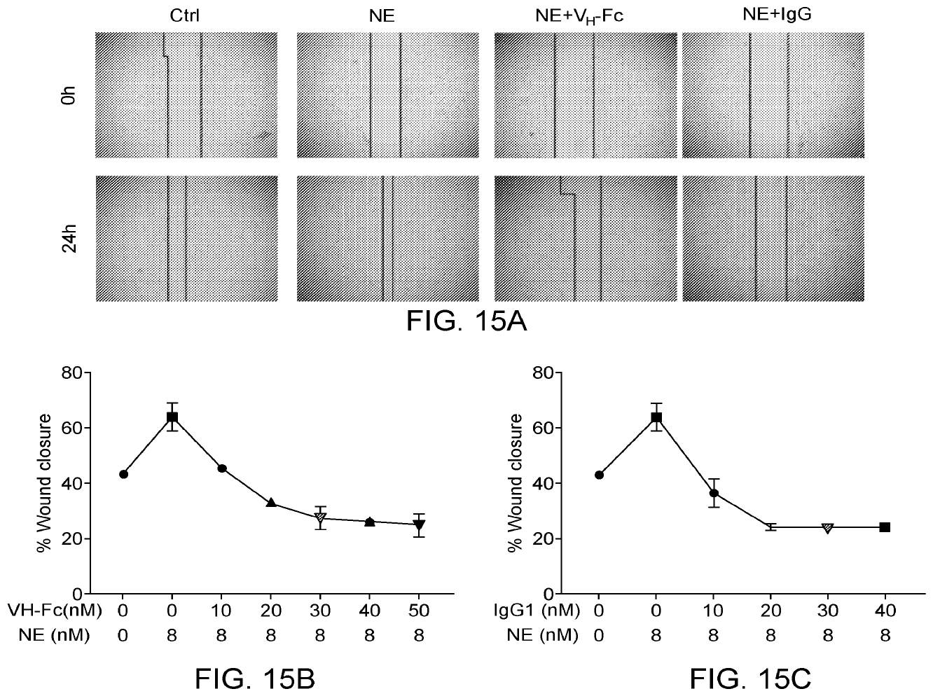

To study the function of the binders, the inhibition effect on neutrophil elastase enzyme activity was tested. The estimated IC₅₀ of VH 1D1.43 was about 60 μM, but after being converting to the VH-Fc form, the IC₅₀ was 5.3±1.2 μM, a 10-fold elevation compared to the VH form (FIGS. 13A and 13C). Additionally, the estimated IC₅₀ of Fab 1C10 was about 5 μM. After converting it to an IgG1 form, the IC₅₀ was elevated to 1.8±0.9 μM (FIGS. 13B and 13D). In addition, the effects of binders on the uptake of NE by cancer cells was also tested as NE needs to enter the cytoplasm to stimulate cell proliferation. First, the uptake ability of three different cancer cells on NE was tested. The results demonstrated that, compared with the A549 cell and SK-BR-3 cell, PC-3 cells exhibited the highest ability to uptake NE. To test the blocking function of binders on NE uptake by cancer cells, IgG1 1C10 and VH-Fc 1D1.43 were incubated with NE at room temperature for 20 minutes, separately, then the mixture was added to PC-3 tumor cells followed by another 24 hour incubation. The results demonstrated that both IgG1 1C10 and VH-Fc 1D1.43 could significantly decrease the NE uptake by cancer cells (FIG. 14), and the inhibition abilities were comparable. As NE plays a similar role in fibroblast proliferation, it was tested whether NE binders could inhibit the fibroblast differentiation promoted by NE. A wound-healing assay demonstrated that NE treatment could facilitate the wound closure, and the function of NE was inhibited by IgG1 1C10 and VH-Fc 1D1.43. The ratio of NE-Abs for inhibiting the differentiation of the fibroblast induced by NE was about 1:1.25 (FIG. 15). These results demonstrated that IgG1 1C10 and VH-Fc 1D1.43 are potent antibody-based inhibitors of neutrophil elastase and can be used to treat cancer and inflammatory-related diseases.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention claimed is:

1. An antibody comprising:
(i) a heavy chain variable domain or region comprising the amino acid sequences set forth in SEQ ID NO:1 or SEQ ID NO: 1 with one, two, or three amino acid additions, deletions, or substitutions, SEQ ID NO:2 or SEQ ID NO:2 with one, two, or three amino acid additions, deletions, or substitutions, and SEQ ID NO:3 or SEQ ID NO:3 with one, two, or three amino acid additions, deletions, or substitutions, and a light chain variable domain or region comprising the amino acid sequences set forth in SEQ ID NO:9 or SEQ ID NO:9 with one, two, or three amino acid additions, deletions, or substitutions, SEQ ID NO:10 or SEQ ID NO:10 with one, two, or three amino acid additions, deletions, or substitutions, and SEQ ID NO:11 or SEQ ID NO:11 with one, two, or three amino acid additions, deletions, or substitutions; or
(ii) a heavy chain variable domain or region comprising the amino acid sequences set forth in SEQ ID NO:17 or SEQ ID NO:17 with one, two, or three amino acid additions, deletions, or substitutions, SEQ ID NO:18 or SEQ ID NO:18 with one, two, or three amino acid additions, deletions, or substitutions, and SEQ ID NO:19 or SEQ ID NO:19 with one, two, or three amino acid additions, deletions, or substitutions.

2. The antibody of claim 1, wherein said antibody comprises the ability to bind to SEQ ID NO:74, SEQ ID NO:75, or SEQ ID NO:166.

3. The antibody of claim 1, wherein said antibody comprises said heavy chain variable domain or region of said (i).

4. The antibody of claim 3, wherein said heavy chain variable domain or region comprises an amino acid sequence having at least 90 percent identity to the amino acid sequence set forth in SEQ ID NO:8.

5. The antibody of claim 1, wherein said antibody comprises said light chain variable domain or region of said (i).

6. The antibody of claim 5, wherein said light chain variable domain or region comprises an amino acid sequence having at least 90 percent identity to the amino acid sequence set forth in SEQ ID NO:16.

7. The antibody of claim 1, wherein said antibody comprises said heavy chain variable domain or region of said (ii).

8. The antibody of claim 7, wherein said heavy chain variable domain or region comprises an amino acid sequence having at least 90 percent identity to the amino acid sequence set forth in SEQ ID NO:24.

9. An antigen binding fragment comprising:
(i) a heavy chain variable domain or region comprising the amino acid sequences set forth in SEQ ID NO:1 or SEQ ID NO:1 with one, two, or three amino acid additions, deletions, or substitutions, SEQ ID NO:2 or SEQ ID NO:2 with one, two, or three amino acid additions, deletions, or substitutions, and SEQ ID NO:3 or SEQ ID NO:3 with one, two, or three amino acid additions, deletions, or substitutions, and a light chain variable domain or region comprising the amino acid sequences set forth in SEQ ID NO:9 or SEQ ID NO:9 with one, two, or three amino acid additions, deletions, or substitutions, SEQ ID NO:10 or SEQ ID NO:10 with one, two, or three amino acid additions, deletions, or substitutions, and SEQ ID NO:11 or SEQ ID NO:11 with one, two, or three amino acid additions, deletions, or substitutions; or
(ii) a heavy chain variable domain or region comprising the amino acid sequences set forth in SEQ ID NO:17 or SEQ ID NO:17 with one, two, or three amino acid additions, deletions, or substitutions, SEQ ID NO:18 or SEQ ID NO:18 with one, two, or three amino acid additions, deletions, or substitutions, and SEQ ID NO: 19 or SEQ ID NO:19 with one, two, or three amino acid additions, deletions, or substitutions.

10. The antigen binding fragment of claim 9, wherein said antigen binding fragment comprises the ability to bind to SEQ ID NO:74, SEQ ID NO: 75, or SEQ ID NO:166.

11-20. (canceled)

21. An antibody domain comprising a heavy chain variable domain or region comprising the amino acid sequences set forth in SEQ ID NO:17 or SEQ ID NO:17 with one, two, or three amino acid additions, deletions, or substitutions, SEQ ID NO:18 or SEQ ID NO:18 with one, two, or three amino acid additions, deletions, or substitutions, and SEQ ID NO:19 or SEQ ID NO:19 with one, two, or three amino acid additions, deletions, or substitutions.

22. The antibody domain of claim 21, wherein said antibody domain comprises the ability to bind to SEQ ID NO:74, SEQ ID NO:75, or SEQ ID NO:166.

23. A nucleic acid comprising a nucleic acid sequence encoding at least part of an antibody of claim 1.

24-27. (canceled)

28. A host cell comprising a nucleic acid encoding at least part of an antibody of claim 1.

29. A composition comprising an antibody of claim 1.

30-31. (canceled)

32. A method of treating a mammal having cancer or an inflammatory condition, wherein said method comprises administering, to said mammal, a composition comprising an antibody of claim 1.

33-36. (canceled)

37. A method for binding a binding molecule to an NE polypeptide, wherein said method comprises contacting said NE polypeptide with an antibody of claim 1.

38. (canceled)

39. The method of claim 37, wherein said contacting is performed in vivo.

40. The method of claim 37, wherein said contacting is performed within a mammal by administering said antibody, said antigen binding fragment, or said antibody domain to said mammal.

41. The method of claim 40, wherein said mammal is a human.
